SEQUENCE LISTING (11 sequences)

SEQ ID NO: 1 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 117 aa, Artificial Sequence
  Humanized antibody sequence
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSS

SEQ ID NO: 3 - protein, 113 aa, Artificial Sequence
  Humanized antibody sequence
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIK

SEQ ID NO: 4 - protein, 10 aa, Artificial Sequence
  affinity matured mouse CDR sequence
GYTFTSYWIN

SEQ ID NO: 5 - protein, 17 aa, Artificial sequence
  affinity matured mouse CDR sequence
NIYPGSSLTNYNEKFKN

SEQ ID NO: 6 - protein, 8 aa, Artificial sequence
  affinity matured mouse CDR sequence
LSTGTFAY

SEQ ID NO: 7 - protein, 17 aa, Artificial sequence
  affinity matured mouse CDR sequence
KSSQSLWDSGNQKNFLT

SEQ ID NO: 8 - protein, 7 aa, Artificial sequence
  affinity matured mouse CDR sequence
WTSYRES

SEQ ID NO: 9 - protein, 9 aa, Artificial sequence
  affinity matured mouse CDR sequence
QNDYFYPHT

SEQ ID NO: 10 - protein, 444 aa, Artificial sequence
  Humanized antibody sequence
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 221 aa, Artificial sequence
  Humanized antibody sequence
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2021/0002369 A1
(43) Pub. Date: Jan. 7, 2021

(54) ANTI-PD-1 ANTIBODY COMPOSITIONS

(71) Applicant: PFIZER INC., NEW YORK, NY (US)

(72) Inventors: Syed Saleem AHMED, Chesterfield, MO (US); Bryan Mark BALTHAZOR, Rock Hill, MO (US); Anjali Pramod MEHTA, Wildwood, MO (US); Tihami QURESHI, St. Louis, MO (US)

(73) Assignee: PFIZER INC., NEW YORK, NY (US)

(21) Appl. No.: 16/978,354

(22) PCT Filed: Mar. 4, 2019
(86) PCT No.: PCT/IB2019/051733

§ 371 Date: Sep. 4, 2020

Related U.S. Application Data
(60) Provisional application No. 62/639,587, filed on Mar. 7, 2018.
(60) Provisional application No. 62/807,912, filed on Feb. 20, 2019.

(51) Int. Cl. C07K 16/28; A61K 9/00; A61K 47/26; A61K 47/18

(52) U.S. Cl. CPC C07K 16/2818; A61K 9/0019; A61K 47/26; A61K 45/06; C07K 2317/565; C07K 2317/41; C07K 2317/24; A61K 47/183

(57) ABSTRACT

The present invention relates generally to the field of pharmaceutical formulations of antibodies. Specifically, the present invention relates to a high concentration antibody formulation and its pharmaceutical preparation and use. This invention is exemplified by a formulation of an anti-PD-1 antibody.

### FIELD

The present invention relates to the field of pharmaceutical formulations of antibodies. Specifically, the present invention relates to an anti-PD-1 antibody formulation and its pharmaceutical preparation and use.

### BACKGROUND

Antibody therapeutics are typically administered on a regular basis and generally involve several mg/kg dosing by injection. Parenteral delivery is a common route of administration for therapeutic antibody. Relatively high concentration antibody formulations are desirable for parenteral administration in order to minimize the volume of each dose.

Development of highly concentrated protein formulations can be a challenge due to issues relating to the physical and chemical stability of the protein, manufacture, storage, and delivery of the protein formulation. Increased viscosity of antibody formulations can cause problems from drug manufacture through drug delivery to the patient. Various attempts have been made to study the effect of viscosity-reducing agents on highly concentrated aqueous protein-containing formulations.

It has been shown that anti-PD-1 antibody is useful in the treatment of hyperproliferative disorders, including type 1 cancer. There is a need for a stable, high concentration antibody preparation of an anti-PD-1 antibody having suitable viscosity to meet the medical need of patients suffering from conditions mediated by PD-1, such as cancer.

### SUMMARY

Compositions comprising an anti-PD-1 antibody and excipients capable of reducing the viscosity of a formulation comprising the antibody are provided. It is demonstrated that certain formulations are effective to reduce viscosity. Advantageously, the compositions provided herein demonstrate viscosity behavior suitable to achieve concentrations of greater than 100 mg/mL for a drug product to be used for therapeutic treatment.

Provided herein are pharmaceutical compositions which support high concentrations of bioactive antibody in solution and are suitable for parenteral administration, including subcutaneous, intravenous, intramuscular, intraperitoneal, or intradermal injection. The compositions comprise an anti-PD-1 antibody, a disaccharide, a buffer, a chelating agent, and a polysorbate. In some embodiments the pH of the composition can be between about 4.5 and 5.5. In some embodiments, the composition preferably has a viscosity of between about 1 centiPoise (cP) and about 20 cP. In some embodiments, the preferred route of administration is subcutaneous injection.

In some embodiments, the composition can comprise or consist essentially of between about 100 mg/ml to about 200 mg/ml anti-PD-1 antibody, a disaccharide, a buffer, a chelating agent, and a polysorbate, and has a pH of about 4.5 to about 5.5. In some embodiments, the composition can consist essentially of about 150 mg/ml anti-PD-1 antibody, a disaccharide, a buffer, a chelating agent, and a polysorbate, and has a pH of about about 5.0.

In some embodiments, the composition does not comprise an anti-oxidant. In some embodiments, the composition does not comprise methionine, such as for example without limitation, L-methionine, or a pharmaceutically acceptable salt thereof. In some embodiments, the composition does not comprise arginine.

In some embodiments, the composition may have a viscosity of less than about 50 cP, less than about 40 cP, less than about 30 cP, or less than about 20 cP at 20° C. In some embodiments, the composition may have a viscosity of about 5 to about 50 cP at 20° C. In some embodiments, the composition may have a viscosity of about 5 to about 40 cP at 20° C. In some embodiments, the composition may have a viscosity of about 5 to about 30 cP at 20° C. In some embodiments, the composition may have a viscosity of about 5 to about 20 cP at 20° C. In some embodiments, the composition may have a viscosity of about 10 to about 20 cP at 20° C. In some embodiments, the composition may have a viscosity of about 14 to about 16 cP at 20° C. In some embodiments, the composition may have a viscosity of about 14 cP at 20° C.

In some embodiments, the concentration of polysorbate can be from about 0.01 to about 0.3 mg/ml. In some embodiments, the concentration of polysorbate is about 0.2 mg/ml. In some embodiments, the polysorbate is polysorbate 80.

In some embodiments, the disaccharide can be trehalose. In some embodiments the trehalose is trehalose dihydrate. In some embodiments the concentration of trehalose can be about 1 mg/ml to about 100 mg/ml. In some embodiments, the concentration of trehalose is about 84 mg/ml. In other embodiments, the concentration of trehalose is about 50 mg/ml.

In other embodiments, the disaccharide agent can be sucrose. In some embodiments the concentration of sucrose can be about 1 mg/ml to about 100 mg/ml. In some embodiments, the concentration of sucrose is about 50 mg/ml.

In some embodiments, the buffer can be histidine buffer. In some embodiments, the concentration of histidine buffer can be from about 1.0 to about 30 mM. In some embodiments, the concentration of histidine buffer is about 20 mM histidine.

In other embodiments, the buffer can be acetate buffer. In some embodiments, the concentration of acetate buffer can be from about 1.0 to about 30 mM. In some embodiments, the concentration of acetate buffer is about 20 mM acetate.

In some embodiments, the chelating agent can be EDTA, including for example without limitation disodium EDTA and disodium EDTA dihydrate. In some embodiments, the concentration of EDTA can be from about 0.01 to about 0.3 mg/mL. In some embodiments, the concentration of EDTA can be from about 0.01 mg/mL, about 0.05 mg/mL, about 0.1 mg/mL, about 0.15 mg/mL, about 0.2 mg/mL, about 0.25 mg/mL, or about 0.3 mg/mL. In some embodiments, the concentration of EDTA is about 0.04, about 0.045, or about 0.05 mg/mL.

In some embodiments, the antibody concentration can be between about 100 mg/ml to about 150 mg/ml. In some embodiments, the antibody concentration can be about 130 mg/ml, about 135 mg/ml and about 140 mg/ml. In some embodiments, the antibody concentration is about 150 mg/ml. In some embodiments, the antibody concentration is about 120 mg/ml.

In some embodiments, the composition can further comprise arginine. In some embodiments, the concentration of arginine is is between about 25mM to about 300mM, preferably about 50 mM, about 100 mM, about 150 mM, about 200 mM, or about 250 mM.

In some embodiments, the composition can further comprise proline. In some embodiments, the concentration of proline is between about 25 mM to about 300 mM, preferably about 100 mM or about 200 mM.

In some embodiments, the composition consists essentially of about 150 mg/ml anti-PD-1 antibody; about 20 mM histidine buffer; about 84 mg/ml trehalose dihydrate; about 0.2 mg/ml PS80; and about 0.05 mg/ml EDTA. In some embodiments, the composition has a pH of 5.0+/−0.5.

In some embodiments, the composition consists essentially of about 150 mg/ml anti-PD-1 antibody; about 20 mM histidine buffer; about 100 mM arginine HCl, about 50 mg/ml trehalose dihydrate; about 0.2 mg/ml PS80; and about 0.05 mg/ml EDTA. In some embodiments, the composition has a pH of 5.0+/−0.5 and a viscosity of about 10 cP to about 16 cP at 20° C. In some embodiments, the composition has a viscosity of about 15 cP at 20° C.

In some embodiments, the composition consists essentially of about 100 mg/ml, about 110 mg/ml, about 120 mg/ml, about 130 mg/ml, about 140 mg/ml, about 145 mg/ml, about 148 mg/ml, about 149 mg/ml, about 150 mg/ml, about 151 mg/ml, or about 152 mg/ml of an antibody, about 20 mM histidine buffer, about 84 mg/ml trehalose dihydrate, about 0.2 mg/ml PS80, about 0.05 mg/ml EDTA, and the composition is of a pH 5.0+/−0.5. In some embodiments, the composition has a viscosity of about 10 cP to about 16 cP at 20° C.

In some embodiments, the composition consists essentially of about 100 mg/ml, about 110 mg/ml, about 120 mg/ml, about 130 mg/ml, about 140 mg/ml, about 145 mg/ml, about 148 mg/ml, about 149 mg/ml, about 150 mg/ml, about 151 mg/ml, or about 152 mg/ml of an anti-PD-1 antibody, about 20 mM histidine buffer, about 84 mg/ml trehalose, about 0.2 mg/ml PS80, about 0.05 mg/ml EDTA, and the composition is of a pH 5.0+/−0.5.

In some embodiments, the composition consists essentially of about 150 mg/ml of an anti-PD-1 antibody, about 20 mM histidine buffer, about 84 mg/ml trehalose dihydrate, about 0.2 mg/ml PS80, about 0.05 mg/ml EDTA, and the composition is of a pH 5.0+/−0.5.

In some embodiments, the antibody can be a human antibody, a humanized antibody, or a chimeric antibody. In some embodiments, the antibody is a monoclonal antibody. In some embodiments, the antibody is of the human IgG₁, IgG₂, IgG_{2Δa}, IgG₃, IgG₄, IgG_{4Δb}, IgG_{4Δc}, IgG₄ S228P, IgG_{4Δb} S228P, and IgG_{4Δc} S228P subclass. In some embodiments, the antibody is of the IgG4 isotype and comprises a stabilized hinge, e.g., S228P.

In some embodiments, the antibody can be PF-06801591, nivolumab, pembrolizumab, cemiplimab, or spartalizumab. In other embodiments, the antibody can be an antigen binding portion of PF-06801591, nivolumab, pembrolizumab, cemiplimab, or spartalizumab.

In some embodiments, the antibody can comprise a heavy chain variable region (VH) comprising a VH complementarity determining region one (CDR1), a VH CDR2, and a VH CDR3 of the VH sequence shown in SEQ ID NO: 2; and/or a light chain variable region (VL) comprising a VL CDR1, a VL CDR2, and a VL CDR3 of the VL sequence shown in SEQ ID NO: 3. In some embodiments, the VH CDR1 comprises the amino acid sequence shown in SEQ ID NO: 4, the VH CDR2 comprises the amino acid sequence shown in SEQ ID NO: 5, and the VH CDR3 comprises the amino acid sequence shown in SEQ ID NO: 6, the VL CDR1 comprises the amino acid sequence shown in SEQ ID NO: 7, the VL CDR2 comprises the amino acid sequence shown in SEQ ID NO: 8, and the VL CDR3 comprises the amino acid sequence shown in SEQ ID NO: 9.

In some embodiments, the VH region comprises the amino acid sequence shown in SEQ ID NO: 2, or a variant with one or several conservative amino acid substitutions in residues that are not within a CDR and/or the VL region comprises the amino acid sequence shown in SEQ ID NO: 3, or a variant thereof with one or several amino acid substitutions in amino acids that are not within a CDR. In some embodiments, the antibody can comprise an amino acid sequence that is at least 90% identical to a heavy chain variable region amino acid sequence shown in SEQ ID NO: 2, and an amino acid sequence that is at least 90% identical to a light chain variable region amino acid sequence shown in SEQ ID NO: 3. In some embodiments, the antibody can comprise a variable heavy chain sequence comprising the amino acid sequence shown in SEQ ID NO: 10 and a variable light chain sequence comprising the amino acid sequence shown in SEQ ID NO: 11. In some embodiments, the antibody comprises a VH region produced by the expression vector with ATCC Accession No. PTA-121183. In some embodiments, the antibody comprises a VL region produced by the expression vector with ATCC Accession No. PTA-121182. In some embodiments, the antibody is an antibody which specifically binds to PD-1 and competes with and/or binds to the same PD-1 epitope as the antibodies as described herein.

In some embodiments, the composition consists essentially of about 150 mg/ml of PF-06801591, about 20 mM histidine buffer, about 84 mg/m 1 trehalose dihydrate, about 0.2 mg/ml PS80, and about 0.05 mg/ml EDTA; wherein the composition is of a pH 5.0+/−0.1 and contains no methionine or argnine. In some embodiments, the composition consists essentially of about 150 mg/ml of nivolumab, about 20 mM histidine buffer, about 84 mg/ml trehalose dihydrate, about 0.2 mg/ml PS80, and about 0.05 mg/ml EDTA; wherein the composition is of a pH 5.0+/−0.1 and contains no methionine or argnine. In some embodiments, the composition consists essentially of about 150 mg/ml of pembrolizumab, about 20 mM histidine buffer, about 84 mg/ml trehalose dihydrate, about 0.2 mg/ml PS80, and about 0.05 mg/ml EDTA; wherein the composition is of a pH 5.0+/−0.1 and contains no methionine or argnine.

In some embodiments, the composition may not be lyophilized. In other embodiments, the composition may be lyophilized.

Also provided herein are methods for treating a condition in a subject, wherein the methods comprise: administering to a subject in need thereof an effective amount of the pharmaceutical composition as described herein. In some embodiments, the condition is a cancer. In some embodiments, the cancer is selected from the group consisting of gastric cancer, sarcoma, lymphoma, leukemia, head and neck cancer, nasopharyngeal cancer, thymic cancer, epithelial cancer, epithelial ovarian cancer, salivary cancer, liver cancer, stomach cancer, thyroid cancer, lung cancer (including for example without limitation non-small cell lung cancer), ovarian cancer, fallopian tube cancer, breast cancer (including for example without limitation triple-negative breast cancer), prostate cancer, esophageal cancer, pancreatic cancer, glioma, leukemia, multiple myeloma, renal cell carcinoma, bladder cancer, cervical cancer, choriocarcinoma, colon cancer, colorectal cancer, oral cancer, skin cancer, peritoneal cancer, and melanoma. In some embodiments, the subject is a previously treated adult patient with locally advanced or metastatic melanoma, squamous cell head and neck cancer (SCHNC), ovarian carcinoma, sarcoma, or relapsed or refractory classic Hodgkin's Lymphoma (cHL). In some embodiments, the cancer can be a platinum resistant and/or platinum refractory cancer, such as, for example, platinum resistant and/or refractory ovarian cancer, platinum resistant and/or refractory breast cancer, or platinum resistant and/or refractory lung cancer.

In some embodiments, an anti-PD-1 antibody pharmaceutical composition provided herein is administered at a dosage of between about 25 mg to about 1000 mg, preferably about 50 mg, about 100 mg, about 125 mg, about 150 mg, about 200 mg, about 250 mg, about 300 mg, about 350 mg, about 400 mg, about 450 mg, about 500 mg, about 525 mg, about 550 mg, about 600 mg, about 650 mg, about 700 mg, about 750 mg, or about 800 mg. In some embodiments, the composition is administered at a dosage of between about 0.5 mg/kg to about 15 mg/kg, preferably about 0.5 mg/kg, about 1.0 mg/kg, about 3.0 mg/kg, about 5.0 mg/kg, or about 10 mg/kg. In some embodiments, the composition is administered once every 7, 14, 21, or 28 days. In some embodiments, the composition is administered subcutaneously. In other embodiments, the composition is administered intravenously. In some embodiments, the composition is administered as a single 2 ml subcutaneous injection. In some embodiments, the composition is administered once every three weeks. In some embodiments, the composition is administered once every four weeks. In some embodiments, the composition is administered at a dose of 300 mg subcutaneously. In some embodiments, the composition is administered as a subcutaneous dose of 300 mg once every 28 days.

Also provided herein are methods of inhibiting tumor growth or progression in a subject who has a tumor, comprising administering to the subject an effective amount of the pharmaceutical composition as described herein.

Also provided herein are methods of inhibiting or preventing metastasis of cancer cells in a subject, comprising administering to the subject in need thereof an effective amount of the pharmaceutical composition as described herein.

Also provided herein are methods of inducing tumor regression in a subject who has a PD-1 expressing tumor, comprising administering to the subject an effective amount of the pharmaceutical composition as described herein.

In some embodiments, the antibody herein can be administered parenterally in a subject. In some embodiments, the subject is a human.

In some embodiments, the method can further comprise administering an effective amount of at least one other therapeutic agent. In some embodiments, the therapeutic agent is, for example, crizotinib, palbociclib, talazoparib, an anti-CTLA4 antibody, an anti-4-1BB antibody, an anti-OX40 antibody, a second PD-1 antibody, a CAR-T cell, or a chemotherapeutic agent.

Also provided herein are uses of any of the anti-PD-1 antibody pharmaceutical compositions provided herein in the manufacture of a medicament for the treatment of cancer or for inhibiting tumor growth or progression in a subject in need thereof.

Also provided are anti-PD-1 antibody pharmaceutical compositions for use in the treatment of a cancer or for inhibiting tumor growth or progression in a subject in need thereof. In some embodiments, the cancer is, for example without limitation, gastric cancer, sarcoma, lymphoma, Hodgkin's lymphoma, leukemia, head and neck cancer, thymic cancer, epithelial cancer, salivary cancer, liver cancer, stomach cancer, thyroid cancer, lung cancer (including, for example, non-small-cell lung carcinoma), ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma.

Also provided herein are methods for enhancing the immunogenicity or therapeutic effect of a vaccine for the treatment of a cancer in a mammal, particularly a human, which method comprises administering to the mammal receiving the vaccine an effective amount of an anti-PD-1 antibody composition provided by the present disclosure.

Also provided herein are methods for treating a cancer in a mammal, particularly a human, which method comprises administering to the mammal (1) an effective amount of a vaccine capable of eliciting an immune response against cells of the cancer and (2) an effective amount of an anti-PD-1 antibody pharmaceutical composition provided by the present disclosure. In some embodiments, the composition is administered as a subcutaneous bilateral dose of about 125 to about 300 mg. In some embodiments, the composition is administered as a subcutaneous bilateral dose of 300 mg.

### BRIEF DESCRIPTION OF THE DRAWINGS

FIG. 1 depicts a graph comparing the viscosity of anti-PD-1 antibody formulations at different pH values.

FIG. 2 depicts a graph comparing the viscosity of anti-PD-1 antibody formulations with varying concentrations of arginine HCl.

FIG. 3 depicts a graph comparing the viscosity of anti-PD-1 antibody formulation at different pH values.

FIG. 4 depicts a graph comparing the viscosity of anti-PD-1 antibody formulations with 100 mM arginine HCl addition, with or without proline.

FIG. 5 depicts a graph comparing the viscosity of anti-PD-1 antibody formulations 7 and 8.

FIG. 6 depicts a graph showing the glycan heterogeneity in anti-PD-1 antibody mAb7.

FIG. 7 depicts a graph showing the thermal properties of various anti-PD-1 antibodies in formulation 7.

### DETAILED DESCRIPTION

Disclosed herein are compositions having reduced viscosity. Advantageously, the compositions stably support high concentrations of bioactive antibody in solution and are suitable for parenteral administration, including or subcutaneous, intravenous, intramuscular, intraperitoneal, or intradermal injection.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning:

A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Cabs, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### Definitions

The following terms, unless otherwise indicated, shall be understood to have the following meanings: the term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

As used herein, the terms “formulation” or “composition” as they relate to an antibody are meant to describe the antibody in combination with a pharmaceutically acceptable excipient comprising at least one tonicity agent, at least one buffer, at least one chelating agent, at least one surfactant, wherein the pH is as defined.

The terms “pharmaceutical composition” or “pharmaceutical formulation” refer to preparations which are in such form as to permit the biological activity of the active ingredients to be effective.

“Pharmaceutically acceptable excipients” (vehicles, additives) are those, which can safely be administered to a subject to provide an effective dose of the active ingredient employed. The term “excipient” or “carrier” as used herein refers to an inert substance, which is commonly used as a diluent, vehicle, preservative, binder or stabilizing agent for drugs. As used herein, the term “diluent” refers to a pharmaceutically acceptable (safe and non-toxic for administration to a human) solvent and is useful for the preparation of the liquid formulations herein. Exemplary diluents include, but are not limited to, sterile water and bacteriostatic water for injection (BWFI).

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site. Antigen binding portions include, for example, Fab, Fab′, F(ab′)₂, Fd, Fv, domain antibodies (dAbs, e.g., shark and camelid antibodies), fragments including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR (i.e., in the framework region), appropriate amino acid substitution, preferably, conservative amino acid substitution, can be identified by comparing the subject variable region to the variable regions of other antibodies which contain CDR1 and CDR2 sequences in the same canonincal class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined of any of these approaches. For any given embodiment containing more than one CDR, the CDRs may be defined in accordance with any of Kabat, Chothia, extended, AbM, contact, and/or conformational definitions.

As known in the art, a “constant region” of an antibody refers to the constant region of the antibody light chain or the constant region of the antibody heavy chain, either alone or in combination.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example. As used herein, “humanized” antibody refers to forms of non-human (e.g. murine) antibodies that are chimeric immunoglobulins, immunoglobulin chains, or fragments thereof (such as Fv, Fab, Fab′, F(ab′)2 or other antigen-binding subsequences of antibodies) that contain minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen binding residues.

As used herein, the term “human antibody” is intended to include antibodies having variable and constant regions derived from human germline immunoglobulin sequences. This definition of a human antibody includes antibodies comprising at least one human heavy chain polypeptide or at least one human light chain polypeptide. The human antibodies of the invention may include amino acid residues not encoded by human germ line immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germ line of another mammalian species, such as a mouse, have been grafted onto human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, “humanized” antibody refers to forms of non-human (e.g. murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. Preferably, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Other forms of humanized antibodies have one or more CDRs (CDR L1, CDR L2, CDR L3, CDR H1, CDR H2, or CDR H3) which are altered with respect to the original antibody, which are also termed one or more CDRs “derived from” one or more CDRs from the original antibody.

As used herein, the term “mAb7” is used to refer to an anti-PD-1 antibody comprising the amino acid sequence of the heavy chain and light chain variable regions shown in SEQ ID NO: 2 and SEQ ID NO: 3, respectively.

mAb7 heavy chain variable region amino
acid sequence:
(SEQ ID NO: 2)
QVQLVQSGAEVKKPGASVKVSCKASGYTFTSYWINWVRQAPGQGLEWMGN
IYPGSSLTNYNEKFKNRVTMTRDTSTSTVYMELSSLRSEDTAVYYCARLS
TGTFAYWGQGTLVTVSS
mAb7 light chain variable region amino
acid sequence:
(SEQ ID NO: 3)
DMMTQSPDSLAVSLGERATINCKSSQSLWDSGNQKNFLTWYQQKPGQPPK
LLIYWTSYRESGVPDRFSGSGSGTDFTLTISSLQAEDVAVYYCQNDYFYP
HTFGGGTKVEIK

The generation and characterization of mAb7 is described in the Examples of WO2016/092419, the entire content of which is herein incorporated by reference. In some embodiments, the term “mAb7” refers to immunoglobulin encoded by (a) a polynucleotide encoding mAb7 light chain variable region that has a deposit number of ATCC No. PTA-121182, and (b) a polynucleotide encoding mAb7 heavy chain variable region that has a deposit number of ATCC No. PTA-121183.

The term “epitope” refers to that portion of a molecule capable of being recognized by and bound by an antibody at one or more of the antibody's antigen-binding regions. Epitopes often consist of a surface grouping of molecules such as amino acids or sugar side chains and have specific three-dimensional structural characteristics as well as specific charge characteristics. In some embodiments, the epitope can be a protein epitope. Protein epitopes can be linear or conformational. In a linear epitope, all of the points of interaction between the protein and the interacting molecule (such as an antibody) occur linearly along the primary amino acid sequence of the protein. A “nonlinear epitope” or “conformational epitope” comprises noncontiguous polypeptides (or amino acids) within the antigenic protein to which an antibody specific to the epitope binds. The term “antigenic epitope” as used herein, is defined as a portion of an antigen to which an antibody can specifically bind as determined by any method well known in the art, for example, by conventional immunoassays. Once a desired epitope on an antigen is determined, it is possible to generate antibodies to that epitope, e.g., using the techniques described in the present specification. Alternatively, during the discovery process, the generation and characterization of antibodies may elucidate information about desirable epitopes. From this information, it is then possible to competitively screen antibodies for binding to the same epitope. An approach to achieve this is to conduct competition and cross-competition studies to find antibodies that compete or cross-compete with one another for binding to PD-1, e.g., the antibodies compete for binding to the antigen.

As used herein, the terms “isolated antibody” or “purified antibody” refers to an antibody that by virtue of its origin or source of derivation has one to four of the following: (1) is not associated with naturally associated components that accompany it in its native state, (2) is free of other proteins from the same species, (3) is expressed by a cell from a different species, or (4) does not occur in nature.

An antibody is “substantially pure,” “substantially homogeneous,” or “substantially purified” when at least about 60 to 75% of a sample exhibits a single species of antibody. A substantially pure antibody can typically comprise about 50%, 60%, 70%, 80% or 90% w/w of an antibody sample, more usually about 95%, and preferably will be over 99% pure. Antibody purity or homogeneity may be tested by a number of means well known in the art, such as polyacrylamide gel electrophoresis or HPLC.

The term “antibody” refers to an antibody that binds to a target and prevents or reduces the biological effect of that target. In some embodiments, the term can denote an antibody that prevents the target, e.g., PD-1, to which it is bound from performing a biological function.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a PD-1 epitope is an antibody that binds this epitope sequence with greater affinity, avidity, more readily, and/or with greater duration than it binds to other sequences. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, “immunospecific” binding of antibodies refers to the antigen specific binding interaction that occurs between the antigen-combining site of an antibody and the specific antigen recognized by that antibody (i.e., the antibody reacts with the protein in an ELISA or other immunoassay, and does not react detectably with unrelated proteins).

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

As used herein, the term “PD-1” refers to any form of PD-1 and variants thereof that retain at least part of the activity of PD-1. Unless indicated differently, such as by specific reference to human PD-1, PD-1 includes all mammalian species of native sequence PD-1, e.g., human, canine, feline, equine, and bovine. One exemplary human PD-1 is found as Uniprot Accession Number Q15116 (SEQ ID NO: 1).

(SEQ ID NO: 1)
MQIPQAPWPV VWAVLQLGWR PGWFLDSPDR PWNPPTFSPA
LLVVTEGDNA TFTCSFSNTS ESFVLNWYRM SPSNQTDKLA
AFPEDRSQPG QDCRFRVTQL PNGRDFHMSV VRARRNDSGT
YLCGAISLAP KAQIKESLRA ELRVTERRAE VPTAHPSPSP
RPAGQFQTLV VGVVGGLLGS LVLLVWVLAV ICSRAARGTI
GARRTGQPLK EDPSAVPVFS VDYGELDFQW REKTPEPPVP
CVPEQTEYAT IVFPSGMGTS SPARRGSADG PRSAQPLRPE
DGHCSWPL.

As used herein, an “anti-PD-1 antibody” refers to an antibody that is able to inhibit PD-1 biological activity and/or downstream events(s) mediated by PD-1. Anti-PD-1 antibodies encompass antibodies that block, antagonize, suppress or reduce (to any degree including significantly) PD-1 biological activity, including downstream events mediated by PD-1, such PD-L1 binding and downstream signaling, PD-L2 binding and downstream signaling, inhibition of T cell proliferation, inhibition of T cell activation, inhibition of IFN secretion, inhibition of IL-2 secretion, inhibition of TNF secretion, induction of IL-10, and inhibition of anti-tumor immune responses. For purposes of the present invention, it will be explicitly understood that the term “anti-PD-1 antibody” (interchangeably termed “PD-1 antibody”) encompasses all the previously identified terms, titles, and functional states and characteristics whereby PD-1 itself, a PD-1 biological activity, or the consequences of the biological activity, are substantially nullified, decreased, or neutralized in any meaningful degree. In some embodiments, an anti-PD-1 antibody binds PD-1 and upregulates an anti-tumor immune response. Examples of anti-PD-1 antibodies are provided herein.

The term “identity” refers to the percent “identity” of two amino acid sequences or of two nucleic acid sequences. The percent identity is generally determined by aligning the sequences for optimal comparision purposes (e.g. gaps can be introduced in the first sequence for best alignment with the second sequence) and comparing the amino acid residues or nucleotides at corresponding positions. The “best alignment” is an alignment of two sequences that results in the highest percent identity. The percent identity is determined by comparing the number of identical amino acid residues or nucleotides within the sequences (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al (1990) J. Mol. Biol. 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucliec Acids Res. 25:3389-3402. Alternatively, PSI-Blast can be used to perform an iterated search that detects distant relationships between molecules (Id.) When utilizing BLAST, Gapped BLAST, and PSI-Blast programs, the default parameters of the respective programs (e.g., XBLAST and NBLAST) can be used. See http://www.ncbi.nlm.nih.gov. Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search.

As used herein, “treatment” is an approach for obtaining beneficial or desired clinical results. For purposes of this invention, beneficial or desired clinical results include, but are not limited to, one or more of the following: reducing the proliferation of (or destroying) neoplastic or cancerous cells, inhibiting metastasis of neoplastic cells, shrinking or decreasing the size of a tumor, remission of cancer, decreasing symptoms resulting from cancer, increasing the quality of life of those suffering from cancer, decreasing the dose of other medications required to treat cancer, delaying the progression of cancer, curing a cancer, and/or prolong survival of patients having cancer.

As used herein, an “effective dosage” or “effective amount” of drug, compound, or pharmaceutical composition is an amount sufficient to effect any one or more beneficial or desired results. In more specific aspects, an effective amount prevents, alleviates or ameliorates symptoms of disease, and/or prolongs the survival of the subject being treated. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing one or more symptoms of a disease such as, for example, cancer including, for example without limitation, gastric cancer, sarcoma, lymphoma, Hodgkin's lymphoma, leukemia, head and neck cancer, squamous cell head and neck cancer, thymic cancer, epithelial cancer, salivary cancer, liver cancer, stomach cancer, thyroid cancer, lung cancer, ovaricancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma, decreasing the dose of other medications required to treat the disease, enhancing the effect of another medication, and/or delaying the progression of the cancer in patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, the term “subject” for purposes of treatment includes any subject, and preferably is a subject who is in need of the treatment of the targeted pathologic condition for example autoimmune disease. For purposes of prevention, the subject is any subject, and preferably is a subject that is at risk for, or is predisposed to, developing the targeted pathologic condition. The term “subject” is intended to include living organisms, e.g., prokaryotes and eukaryotes. Examples of subjects include mammals, e.g., humans, dogs, cows, horses, pigs, sheep, goats, cats, mice, rabbits, rats, and transgenic non-human animals. In specific embodiments of the invention, the subject is a human.

As used herein, the term “polynucleotide” or “nucleic acid”, used interchangeably herein, means a polymeric form of nucleotides either ribonucleotides or deoxynucleotides or a modified form of either type of nucleotide and may be single and double stranded forms. A “polynucleotide” or a “nucleic acid” sequence encompasses its complement unless otherwise specified. As used herein, the term “isolated polynucleotide” or “isolated nucleic acid” means a polynucleotide of genomic, cDNA, or synthetic origin or some combination thereof, which by virtue of its origin or source of derivation, the isolated polynucleotide has one to three of the following: (1) is not associated with all or a portion of a polynucleotide with which the “isolated polynucleotide” is found in nature, (2) is operably linked to a polynucleotide to which it is not linked in nature, or (3) does not occur in nature as part of a larger sequence.

As used herein, the term “chelating agent” is an excipient that can form at least one bond (e.g., covalent, ionic, or otherwise) to a metal ion. A chelating agent is typically a multidentate ligand that can be used in compositions as a stabilizer to complex with species, which might otherwise promote instability.

As used herein, the term “buffer” refers to an added composition that allows a liquid antibody formulation to resist changes in pH, typically by action of its acid-base conjugate components. When a concentration of a buffer is referred to, it is intended that the recited concentration represent the molar concentration of the free acid or free base form of the buffer.

“Viscosity,” as used herein, may be “absolute viscosity” or “kinematic viscosity.” “Absolute viscosity,” sometimes called dynamic or simple viscosity, is a quantity that describes a fluid's resistance to flow. “Kinematic viscosity” is the quotient of absolute viscosity and fluid density. Kinematic viscosity is frequently reported when characterizing the resistive flow of a fluid using a capillary viscometer. When two fluids of equal volume are placed in identical capillary viscometers and allowed to flow by gravity, a viscous fluid takes longer than a less viscous fluid to flow through the capillary. If one fluid takes 200 seconds to complete its flow and another fluid takes 400 seconds, the second fluid is twice as viscous as the first on a kinematic viscosity scale. If both fluids have equal density, the second fluid is twice as viscous as the first on an absolute viscosity scale. The dimensions of kinematic viscosity are L²/T where L represents length and T represents time. The SI units of kinematic viscosity are m²/s. Commonly, kinematic viscosity is expressed in centistokes, cSt, which is equivalent to mm²/s. The dimensions of absolute viscosity are M/L/T, where M represents mass and L and T represent length and time, respectively. The SI units of absolute viscosity are Pa·s, which is equivalent to kg/m/s. The absolute viscosity is commonly expressed in units of centiPoise, cP, which is equivalent to milliPascal-second, mPa·s.

As used herein, the terms “tonicity agent” or “tonicifier” refers to an excipient that can adjust the osmotic pressure of a liquid antibody formulation. In certain embodiments, the tonicity agent can adjust the osmotic pressure of a liquid antibody formulation to isotonic so that the antibody formulation is physiologically compatible with the cells of the body tissue of the subject. In still other embodiments, the “tonicity agent” may contribute to an improvement in stability of antibodies described herein. An “isotonic” formulation is one that has essentially the same osmotic pressure as human blood. Isotonic formulations generally have an osmotic pressure from about 250 to 350 mOsm. The term “hypotonic” describes a formulation with an osmotic pressure below that of human blood. Correspondingly, the term “hypertonic” is used to describe a formulation with an osmotic pressure above that of human blood, Isotonicity can be measured using a vapor pressure or ice-freezing type osmometer, for example. The tonicity agent can be in an enantiomeric (e.g., L- or D-enantiomer) or racemic form; isomers such as alpha or beta, including alpha, alpha; or beta, beta; or alpha, beta; or beta, alpha; a free acid or free base form; a hydrated form (e.g., monohydrate), or an anhydrous form.

As used herein, the term “polyol” refers an excipient with multiple hydroxyl groups, and includes sugars (reducing and nonreducing sugars), sugar alcohols and sugar acids.

As used herein, the term “surfactant” refers to an excipient that can alter the surface tension of a liquid antibody formulation. In certain embodiments, the surfactant reduces the surface tension of a liquid antibody formulation. In still other embodiments, the “surfactant” may contribute to an improvement in stability of any of the antibody in the formulation. The surfactant may reduce aggregation of the formulated antibody and/or minimize the formation of particulates in the formulation and/or reduces adsorption. The surfactant may also improve stability of the antibody during and after a freeze/thaw cycle.

As used herein, the term “saccharide” refers to a class of molecules that are derivatives of polyhydric alcohols. Saccharides are commonly referred to as carbohydrates and may contain different amounts of sugar (saccharide) units, e.g., monosaccharides, disaccharides and polysaccharides.

As used herein, the term “reducing sugar” is one which contains a hem iacetal group that can reduce metal ions or react covalently with lysine and other amino groups in proteins and a “nonreducing sugar” is one which does not have these properties of a reducing sugar.

A “Iyoprotectant” is a molecule which, when combined with a protein of interest, significantly prevents or reduces physicochemical instability of the protein upon lyophilization and subsequent storage. Exemplary lyoprotectants include sugars and their corresponding sugar alcohols; an amino acid such as monosodium glutamate or histidine; a methylamine such as betaine; a lyotropic salt such as magnesium sulfate; a polyol such as trihydric or higher molecular weight sugar alcohols, e.g., glycerin, dextran, erythritol, glycerol, arabitol, xylitol, sorbitol, and mannitol; propylene glycol; polyethylene glycol; Pluronics®; and combinations thereof. Additional exemplary lyoprotectants include glycerin and gelatin, and the sugars mellibiose, melezitose, raffinose, mannotriose and stachyose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Preferred sugar alcohols are monoglycosides, especially those compounds obtained by reduction of disaccharides such as lactose, maltose, lactulose and maltulose. The glycosidic side group can be either glucosidic or galactosidic. Additional examples of sugar alcohols are glucitol, maltitol, lactitol and iso-maltulose. The preferred lyoprotectant are the non-reducing sugars trehalose or sucrose.

The lyoprotectant is added to the pre-lyophilized formulation in a “lyoprotecting amount” which means that, following lyophilization of the protein in the presence of the lyoprotecting amount of the lyoprotectant, the protein essentially retains its physicochemical stability upon lyophilization and storage.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline, normal (0.9%) saline, or 5% dextrose. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

“Reducing incidence” means any of reducing severity (which can include reducing need for and/or amount of (e.g., exposure to) other drugs and/or therapies generally used for this condition. As is understood by those skilled in the art, individuals may vary in terms of their response to treatment, and, as such, for example, a “method of reducing incidence” reflects administering the anti-PD-1 antibody based on a reasonable expectation that such administration may likely cause such a reduction in incidence in that particular individual.

“Ameliorating” means a lessening or improvement of one or more symptoms as compared to not administering an anti-PD-1 antibody. “Ameliorating” also includes shortening or reduction in duration of a symptom.

Reference to “about” a value or parameter herein includes (and describes) embodiments that are directed to that value or parameter per se. For example, description referring to “about X” includes description of “X.” Numeric ranges are inclusive of the numbers defining the range.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

When introducing elements of the present invention or the preferred embodiment(s) thereof, the articles “a”, “an”, “the” and “said” are intended to mean that there are one or more of the elements. The terms “comprising”, “comprise”, “comprises”, “including” and “having” are intended to be inclusive and mean that there may be additional elements other than the listed elements. It is understood that wherever embodiments are described herein with the language “comprising,” otherwise analogous embodiments described in terms of “consisting of” and/or “consisting essentially of” are also provided.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular.

Exemplary methods and materials are described herein, although methods and materials similar or equivalent to those described herein can also be used in the practice or testing of the present invention. The materials, methods, and examples are illustrative only and not intended to be limiting.

### Anti-PD-1 Antibody Compositions

In one aspect, the invention provides a formulation comprising an anti-PD-1 antibody, the formulation having viscosity of between about 1 cP and about 25 cP. In another aspect, a method is provided for reducing the viscosity of an anti-PD-1 antibody-containing formulation, wherein the method comprises the step of adding to the formulation a viscosity reducing amount of a compound that is capable of reducing the viscosity of an aqueous formulation comprising said anti-PD-1 antibody. The formulation may be in either aqueous or lyophilized form. In aqueous form, the formulation may have a viscosity of no greater than about 150 cP, preferably no greater than about 120 cP, preferably no greater than about 100 cP, preferably no greater than about 90 cP, preferably no greater than about 80 cP, preferably no greater than about 70 cP, preferably no greater than about 60 cP, preferably no greater than about 50 cP, preferably no greater than about 40 cP, preferably no greater than about 30 cP, preferably no greater than about 20 cP, preferably no greater than about 10 cP, preferably no greater than about 5 cP. In some embodiments the composition comprising antibody has a viscosity of between about 1 cP and about 500 cP, between about 1 cP and 200 cP, between about 1 cP and about 150 cP, between about 1 cP and about 100 cP, between about 1 cP and about 90 cP, between about 1 cP and about 80 cP, between about 1 cP and about 70 cP, between about 1 cP and about 60 cP, between about 1 cP and about 50 cP, between about 1 cP and about 40 cP, between about 1 cP and about 30 cP, between about 1 cP and about 20 cP, or between about 1 cP and about 10 cP at 20° C. In some embodiments, the formulation has a viscosity of about 120 cP, about about 115 cP, 110 cP, about 105 cP, about 100 cP, about 95 cP, about 90 cP, about 85 cP, about 80 cP, about 75 cP, about 70 cP, about 65 cP, about 60 cP, about about 55 cP, 50 cP, about 45 cP, about 40 cP, about 35 cP, about 30 cP, about 25 cP, about 20 cP, about 15 cP, or about 10 cP, or about 5 cP. In some embodiments, the formulation has a viscosity of between about 10 cP and 50 cP, between about 10 cP and 100 cP, between about 20 cP and 60 cP, between about 30 cP and 60 cP, between about 40 cP and 60 cP, or between about 50 cP and 60 cP. In some embodiments, in aqueous form, the formulation may have a viscosity of between about 1 cP and 10 cP. In some embodiments, in aqueous form, the formulation may have a viscosity of between about 1 cP and 15 cP. In some embodiments, in aqueous form, the formulation may have a viscosity of between about 1 cP and 20 cP.

Another aspect of the present invention is directed to an article of manufacture comprising a container holding any of the herein described formulations.

In some embodiments, the formulation comprises at least one anti-PD-1 antibody. In some embodiments, more than one antibody may be present. At least one, at least two, at least three, at least four, at least five, or more, different antibodies can be present. Generally, the two or more different antibodies have complementary activities that do not adversely affect each other. The, or each, antibody can also be used in conjunction with other agents that serve to enhance and/or complement the effectiveness of the antibodies. The antibody may be present in the formulation at a concentration ranging from about 0.1 to about 300 mg/ml. In some embodiments the concentration of antibody is about 0.5 mg/ml, about 1 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, about 41 mg/ml, about 42 mg/ml, about 43 mg/ml, about 44 mg/ml, about 45 mg/ml, about 46 mg/ml, about 47 mg/ml, about 48 mg/ml, about 49 mg/ml, about 50 mg/ml, about 51 mg/ml, about 52 mg/ml, about 53 mg/ml, about 54 mg/ml, about 55 mg/ml, about 56 mg/ml, about 57 mg/ml, about 58 mg/ml, about 59 mg/ml, about 60 mg/ml, about 70 mg/ml, about 80 mg/ml, about 90 mg/ml, about 100 mg/ml, about 101 mg/ml, about 102 mg/ml, about 102.5 mg/ml, about 103 mg/ml, about 103.5 mg/ml, about 104 mg/ml, about 104.5 mg/ml, about 105 mg/ml, about 105.5 mg/ml, about 106 mg/ml, about 106.5 mg/ml, about 107 mg/ml, about 107.5 mg/ml, about 108 mg/ml, about 108.5 mg/ml, about 109 mg/ml, about 109.5 mg/ml, about 110 mg/ml, about 111 mg/ml, about 112 mg/ml, about 113 mg/ml, about 114 mg/ml, about 115 mg/ml, about 116 mg/ml, about 117 mg/ml, about 118 mg/ml, about 119 mg/ml, about 120 mg/ml, about 121 mg/ml, about 122 mg/ml, about 123 mg/ml, about 124 mg/ml, about 125 mg/ml, about 126 mg/ml, about 127 mg/ml, about 128 mg/ml, about 129 mg/ml, about 130 mg/ml, about 131 mg/ml, about 132 mg/ml, about 133 mg/ml, about 134 mg/ml, about 135 mg/ml, about 136 mg/ml, about 137 mg/ml, about 138 mg/ml, about 139 mg/ml, about 140 mg/ml, about 141 mg/ml, about 142 mg/ml, about 143 mg/ml, about 144 mg/ml, about 145 mg/ml, about 146 mg/ml, about 147 mg/ml, about 148 mg/ml, about 149 mg/ml, about 150 mg/ml, about 151 mg/ml, about 152 mg/ml, about 153 mg/ml, about 154 mg/ml, about 155 mg/ml, about 156 mg/ml, about 157 mg/ml, about 158 mg/ml, about 159 mg/ml, about 160 mg/ml, about 170 mg/ml, about 180 mg/ml, about 190 mg/ml, about 200 mg/ml, about 201 mg/ml, about 202 mg/ml, about 202.5 mg/ml, about 203 mg/ml, about 203.5 mg/ml, about 204 mg/ml, about 204.5 mg/ml, about 205 mg/ml, about 205.5 mg/ml, about 206 mg/ml, about 206.5 mg/ml, about 207 mg/ml, about 207.5 mg/ml, about 208 mg/ml, about 208.5 mg/ml, about 209 mg/ml, about 209.5 mg/ml, about 210 mg/ml, about 211 mg/ml, about 212 mg/ml, about 213 mg/ml, about 214 mg/ml, about 215 mg/ml, about 216 mg/ml, about 217 mg/ml, about 218 mg/ml, about 219 mg/ml, about 220 mg/ml, about 221 mg/ml, about 222 mg/ml, about 223 mg/ml, about 224 mg/ml, about 225 mg/ml, about 226 mg/ml, about 227 mg/ml, about 228 mg/ml, about 229 mg/ml, about 230 mg/ml, about 231 mg/ml, about 232 mg/ml, about 233 mg/ml, about 234 mg/ml, about 235 mg/ml, about 236 mg/ml, about 237 mg/ml, about 238 mg/ml, about 239 mg/ml, about 240 mg/ml, about 241 mg/ml, about 242 mg/ml, about 243 mg/ml, about 244 mg/ml, about 245 mg/ml, about 246 mg/ml, about 247 mg/ml, about 248 mg/ml, about 249 mg/ml, about 250 mg/ml, about 251 mg/ml, about 252 mg/ml, about 253 mg/ml, about 254 mg/ml, about 255 mg/ml, about 256 mg/ml, about 257 mg/ml, about 258 mg/ml, about 259 mg/ml, about 260 mg/ml, about 270 mg/ml, about 280 mg/ml, about 290 mg/ml, or about 300 mg/ml.

According to some embodiments of the present invention the pH can be in the range of about pH 4.0 to 6.0, preferably between about pH 5.0 and of any of about pH 4.5, about 4.6, about 4.7, about 4.8, about 4.9, about 5.0, about 5.1, about 5.2, about 5.3, about 5.4 or about 5.5. Further preferably the pH is in the range selected from between any one of about pH 4.9, 5.0 or 5.1. In some embodiments the pH is pH 5.0+/−0.5. Values of pH in these ranges provide the composition with lower viscosities.

In some embodiments, the formulation may comprise arginine. In some embodiments, the arginine is arginine hydrochloride, or arginine HCl. [Bryan: although Formulation 7 doesn't contain arginine, we include this for alternatives. Same comment applies for the other excipients, etc. not present in formulation 7 but included in this description.] The concentration of the arginine can range from about 0.1 millimolar (mM) to about 200 mM. In some embodiments, the concentration of the arginine is from about 10 mM to about 150 mM, about 50 mM to about 130 mM, about 80 mM to about 120 mM, or about 90 mM to about 110 mM. In some embodiments, the concentration of the arginine is about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, or about 200 mM. In some embodiments, the concentration of the arginine is 100 mM.

In some embodiments, the tonicity agent can comprise a polyol, a saccharide, a carbohydrate, a salt, such as sodium chloride, or mixtures thereof. The polyol can have a molecular weight that, for example without limitation, is less than about 600 kD (e.g., in the range from about 120 to about 400 kD), and can be, for example without limitation, mannitol, trehalose, sorbitol, erythritol, isomalt, lactitol, maltitol, xylitol, glycerol, lactitol, propylene glycol, polyethylene glycol, inositol, or mixtures thereof. The saccharide or carbohydrate can be, for example without limitation, a monosaccharide, disaccharide or polysaccharide, or mixtures of any of the foregoing. The saccharide or carbohydrate can be, for example without limitation, fructose, glucose, mannose, sucrose, sorbose, xylose, lactose, maltose, sucrose, dextran, pullulan, dextrin, cyclodextrins, soluble starch, hydroxyethyl starch, water-soluble glucans, or mixtures thereof. The tonicity agent can comprise a saccharide such as, for example without limitation, a reducing sugar or non reducing sugar or mixtures thereof. The tonicity agent can comprise a saccharide which is a non-reducing sugar such as, for example without limitation, sucrose, trehalose, and mixtures thereof.

The concentration of the tonicity agent in the composition ranges from about 1 mg/ml to about 300 mg/ml, from about 1 mg/ml to about 200 mg/ml, or from about 1 mg/ml to about 100 mg/ml. Preferably the concentration of the tonicity agent in the composition is about 0.5 mg/ml, about 1 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, about 41 mg/ml, about 42 mg/ml, about 43 mg/ml, about 44 mg/ml, about 45 mg/ml, about 46 mg/ml, about 47 mg/ml, about 48 mg/ml, about 49 mg/ml, about 50 mg/ml, about 51 mg/ml, about 52 mg/ml, about 53 mg/ml, about 54 mg/ml, about 55 mg/ml, about 56 mg/ml, about 57 mg/ml, about 58 mg/ml, about 59 mg/ml, about 60 mg/ml, about 65 mg/ml, about 70 mg/ml, about 75 mg/ml, about 80 mg/ml, about 81 mg/ml, about 82 mg/ml, about 83 mg/ml, about 84 mg/ml, about 85 mg/ml, about 86 mg/ml, about 87 mg/ml, about 88 mg/ml, about 89 mg/ml, about 90 mg/ml, about 91 mg/ml, about 92 mg/ml, about 93 mg/ml, about 94 mg/ml, about 95 mg/ml, about 96 mg/ml, about 97 mg/ml, about 98 mg/ml, about 99 mg/ml, about 100 mg/ml, about 101 mg/ml, about 102 mg/ml, about 103 mg/ml, about 104 mg/ml, about 105 mg/ml, about 106 mg/ml, about 107 mg/ml, about 108 mg/ml, about 109 mg/ml, about 110 mg/ml, about 111 mg/ml, about 112 mg/ml, about 113 mg/ml, about 114 mg/ml, about 115 mg/ml, about 116 mg/ml, about 117 mg/ml, about 118 mg/ml, about 119 mg/ml, about 120 mg/ml, about 121 mg/ml, about 122 mg/ml, about 123 mg/ml, about 124 mg/ml, about 125 mg/ml, about 126 mg/ml, about 127 mg/ml, about 128 mg/ml, about 129 mg/ml, about 130 mg/ml, about 131 mg/ml, about 132 mg/ml, about 133 mg/ml, about 134 mg/ml, about 135 mg/ml, about 136 mg/ml, about 137 mg/ml, about 138 mg/ml, about 139 mg/ml, about 140 mg/ml, about 141 mg/ml, about 142 mg/ml, about 143 mg/ml, about 144 mg/ml, about 145 mg/ml, about 146 mg/ml, about 147 mg/ml, about 148 mg/ml, about 149 mg/ml, or about 150 mg/ml.

The surfactant can be, for example without limitation, a polysorbate, poloxamer, triton, sodium dodecyl sulfate, sodium laurel sulfate, sodium octyl glycoside, lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauroam idopropyl-betaine, cocamidopropyl-betaine, linoleamidopropyl-betaine, myristamidopropyl-betaine, palmidopropyl-betaine, isostearamidopropyl-betaine, myristamidopropyl-dimethylamine, palmidopropyl-dimethylamine, isostearamidopropyl-dimethylamine, sodium methyl cocoyl-taurate, disodium methyl oleyl-taurate, dihydroxypropyl PEG 5 linoleammonium chloride, polyethylene glycol, polypropylene glycol, and mixtures thereof. The surfactant can be, for example without limitation, polysorbate 20, polysorbate 21, polysorbate 40, polysorbate 60, polysorbate 61, polysorbate 65, polysorbate 80, polysorbate 81, polysorbate 85, PEG3350 and mixtures thereof. In some embodiments, the surfactant is polysorbate 80 (PS80).

The concentration of the surfactant generally ranges from about 0.01 mg/ml to about 10 mg/ml, from about 0.01 mg/ml to about 5.0 mg/ml, from about 0.01 mg/ml to about 2.0 mg/ml, from about 0.01 mg/ml to about 1.5 mg/ml, from about 0.01 mg/ml to about 1.0 mg/ml, from about 0.01 mg/ml to about 0.5 mg/ml, from about 0.01 mg/ml to about 0.4 mg/ml, from about 0.01 mg/ml to about 0.3 mg/ml, from about 0.01 mg/ml to about 0.2 mg/ml, from about 0.01 mg/ml to about 0.15 mg/ml, from about 0.01 mg/ml to about 0.1 mg/ml, or from about 0.01 mg/ml, to about 0.05 mg/ml. Further preferably the concentration of the surfactant is about 0.5 mg/ml, about 0.05 mg/ml about 0.06 mg/ml about 0.07 mg/ml about 0.08 mg/ml, about 0.09 mg/ml about 0.1 mg/ml about 0.11 mg/ml about 0.12 mg/ml about 0.13 mg/ml about 0.14 mg/ml about 0.15 mg/ml about 0.16 mg/ml about 0.17 mg/ml about 0.18 mg/ml about 0.19 mg/ml, about 0.2 mg/ml. In some embodiments, the concentration of the surfactant is 0.2 mg/ml.

The buffer can be, for example without limitation, acetate, histidine, succinate, gluconate, citrate, acetic acid, phosphate, phosphoric acid, ascorbate, tartartic acid, maleic acid, glycine, lactate, lactic acid, ascorbic acid, imidazole, bicarbonate and carbonic acid, succinic acid, sodium benzoate, benzoic acid, gluconate, edetate, acetate, malate, imidazole, tris, phosphate, and mixtures thereof. In some embodiments, the buffer is a histidine buffer, wherein the histidine can comprise either L-histidine or D-histidine, a solvated form of histidine, a hydrated form (e.g., monohydrate) of histidine, a salt of histidine (e.g., histidine hydrochloride) or an anhydrous form of histidine or a mixture thereof. Preferably, the buffer is an acetate buffer, wherein the acetate can comprise sodium acetate, acetic acid, or a mixture thereof.

The concentration of the buffer can range from about 0.1 millimolar (mM) to about 100 mM. Preferably, the concentration of the buffer is from about 0.5 mM to about 50 mM, further preferably about 1 mM to about 30 mM, more preferably about 1 mM to about 18 mM, increasingly preferably about 1 mM to about 15 mM. Preferably, the concentration of the buffer is about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 30 mM, about 35 mM, about 40 mM, about 45 mM or about 50 mM. In some embodiments, the concentration of the buffer is about 190 mM, about 200 mM, about 210 mM, about 220 mM, about 230 mM, about 240 mM, about 250 mM, about 260 mM, about 270 mM, about 280 mM, about 290, about 300 mM, about 310 mM, or about 320 mM.

In some embodiments, the chelating agent can be selected from the group consisting of aminopolycarboxylic acids, hydroxyaminocarboxylic acids, N-substituted glycines, 2-(2-amino-2-oxocthyl) aminoethane sulfonic acid (BES), deferoxamine (DEF), citric acid, niacinamide, and desoxycholates and mixtures thereof. In some embodiments, chelating agent is selected from the group consisting of ethylenediaminetetraacetic acid (EDTA), diethylenetriamine pentaacetic acid 5 (DTPA), nitrilotriacetic acid (NTA), N-2-acetamido-2-iminodiacetic acid (ADA), bis(am inoethyl)glycolether, N, N, N′, N′-tetraacetic acid (EGTA), trans-diaminocyclohexane tetraacetic acid (DCTA), glutamic acid, and aspartic acid, N-hydroxyethylim inodiacetic acid (HIMDA), N,N-bis-hydroxyethylglycine (bicine) and N-(trishydroxym ethylm ethyl) 10 glycine (tricine), glycylglycine, sodium desoxycholate, ethylenediamine; propylenediamine; diethylenetriamine; triethylenetetraamine (trien), ethylenediaminetetraaceto EDTA; disodium EDTA, EDTA, calcium EDTA oxalic acid, malate, citric acid, citric acid monohydrate, and trisodium citrate-dihydrate, 8-hydroxyquinolate, amino acids, histidine, cysteine, methionine, peptides, polypeptides, and proteins and mixtures thereof. In some embodiments, the chelating agent is selected from the group consisting of salts of EDTA including dipotassium edetate, disodium edetate, edetate calcium disodium, sodium edetate, trisodium edetate, and potassium edetate; and a suitable salt of deferoxamine (DEF) is deferoxamine mesylate (DFM), or mixtures thereof. Chelating agents used in the invention can be present, where possible, as the free acid or free base form or salt form of the compound, also as an anhydrous, solvated or hydrated form of the compound or corresponding salt. After dissolution into a solution at a given pH and at the same concentration of ethylenediaminetetraacetic (EDTA), various salt, free acid, hydrated or anhyrdrous forms of EDTA containing sodium and water are considered equivalent chelating agents. For example, 0.0500 g/L of EDTA disodium dihydrate (FW: 372.3), 0.0393 g/L of EDTA (FW: 292.3), 0.0452 g/L of EDTA disodium (FW: 336.2), 0.0481 g/L of EDTA yrisodium (FW: 358.2), 0.0511 g/L of EDTA Tetrasodium (FW: 380.2), 0.0422 g/L of EDTA Ssodium (FW: 314.2), and 0.0446 g/L of EDTA sodium hydrate (FW: 332.3) all contain 134.4 pM EDTA are considered equivalent chelating agents.

Most preferably the chelating agent is EDTA, such as for example without limitation, disodium EDTA, calcium EDTA, or disodium EDTA dihydrate.

Particularly preferable is disodium EDTA as it provides the composition with an enhanced antibody stability and/or resistance to aggregation.

The concentration of chelating agent generally ranges from about 0.01 mg/ml to about 50 mg/ml, from about 1 mg/ml to about 10.0 mg/ml, from about 5 mg/ml to about 15.0 mg/ml, from about 0.01 mg/ml to about 1.0 mg/ml, or from about 0.03 mg/ml to about 0.5 mg/ml. Further preferably concentration of chelating agent generally ranges from from about 0.01 mM to about 2.0 mM, from about 0.01 mM to about 1.5 mM, from about 0.01 mM to about 0.5 mM, from about 0.01 mM to about 0.4 mM, from about 0.01 mM to about 0.3 mM, from about 0.01 mM to about 0.2 mM, from about 0.01 mM to about 0.15 mM, from about 0.01 mM to about 0.1 mM, from about 0.01 mM to about 0.09 mM, from about 0.01 mM to about 0.08 mM, from about 0.01 mM to about 0.07 mM, from about 0.01 mM to about 0.06 mM, from about 0.01 mM to about 0.05 mM, from about 0.01 mM to about 0.04 mM, from about 0.01 mM to about 0.03 mM, from about 0.01 mM to about 0.02 mM or from about 0.05 mM to about 0.01 mM. Preferably the concentration of chelating agent can be about 0.01 mg/ml, 0.02 mg/ml, 0.03 mg/ml, about 0.04 mg/ml, about 0.05 mg/ml, about 0.06 mg/ml, about 0.07 mg/ml, about 0.10 mg/ml, about 0.20 mg/ml. Further preferably the concentration of chelating agent is about 0.04 mg/ml, about 0.041 mg/ml, about 0.042 mg/ml, about 0.043 mg/ml, about 0.044 mg/ml, about 0.045 mg/ml, about 0.046 mg/ml, about 0.047 mg/ml, about 0.048 mg/ml, about 0.049 mg/ml, about 0.05 mg/ml, about 0.051 mg/ml, about 0.052 mg/ml, about 0.053 mg/ml, about 0.054 mg/ml, about 0.055 mg/ml, or about 0.056 mg/ml. Most preferably, the concentration of chelating agent is about 0.05 mg/ml.

Chelating agents can lower the formation of reduced oxygen species, reduce acidic species (e.g., deamidation) formation, reduce antibody aggregation, and/or reduce antibody fragmentation, and/or reduce antibody oxidation in the compositions of the present invention. Such chelating agents can reduce or prevent degradation of an antibody that is formulated in comparision to the antibody without the protection of a chelating agent.

Unless stated otherwise, the concentrations listed herein are those concentrations at ambient conditions, i.e., at 25° C. and atmospheric pressure.

In preferred embodiments, the formulation comprises no anti-oxidant. For example, preferred embodiments of the compositions provided herein do not comprise methionine, sodium thiosulfate, catalase, or platinum.

In some embodiments, the formulation can comprise an antioxidant agent. In some embodiments the antioxidant is selected from the group comprising, methionine, sodium thiosulfate, catalase, and platinum.

The concentration of antioxidant generally ranges from about 0.01 mg/ml to about 50 mg/ml, from about 0.01 mg/ml to about 10.0 mg/ml, from about 0.01 mg/ml to about 5.0 mg/ml, from about 0.01 mg/ml to about 1.0 mg/ml, or from about 0.01 mg/ml to about 0.02 mg/ml. Preferably the concentration of antioxidant can be about 0.01 mg/ml, 0.02 mg/ml, 0.03 mg/ml, about 0.04 mg/ml, about 0.05 mg/ml, about 0.06 mg/ml, about 0.07 mg/ml, 0.08 mg/ml, 0.09 mg/ml, about 0.10 mg/ml, 0.11 mg/ml, 0.12 mg/ml, 0.13 mg/ml, about 0.14 mg/ml, about 0.15 mg/ml, about 0.16 mg/ml, about 0.17 mg/ml, 0.18 mg/ml, 0.19 mg/ml about 0.20 mg/ml, about 0.25 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml. Most preferably, the concentration of antioxidant is about 0.01 mg/ml.

In some embodiments the formulation can comprise a preservative. Preferably the preservative agent is selected from Phenol, m-cresol, benzyl alcohol, benzalkonium chloride, benzalthonium chloride, phenoxyethanol and methyl paraben.

The concentration of preservative generally ranges from about 0.001 mg/ml to about 50 mg/ml, from about 0.005 mg/ml to about 15.0 mg/ml, from about 0.008 mg/ml to about 12.0 mg/ml or from about 0.01 mg/ml to about 10.0 mg/ml. Preferably the concentration of preservative can be about 0.1 mg/ml, 0.2 mg/ml, 0.3 mg/ml, about 0.4 mg/ml, about 0.5 mg/ml, about 0.6 mg/ml, about 0.7 mg/ml, 0.8 mg/ml, 0.9 mg/ml about 1.0 mg/ml, 2.0 mg/ml, 3.0 mg/ml, about 4.0 mg/ml, about 5.0 mg/ml, about 6.0 mg/ml, about 7.0 mg/ml, 8.0 mg/ml, 9.0 mg/ml about 9.1 mg/ml, about 9.2 mg/ml, 9.3 mg/ml, 9.4 mg/ml, 9.5 mg/ml, 9.6 mg/ml, 9.7 mg/ml, 9.8 mg/ml, 9.9 mg/ml, 10.0 mg/ml. Most preferably, the concentration of preservative is about 0.1 mg/ml or 9.0 mg/mL.

In some embodiments, the composition does not contain an antioxidant.

In some embodiments, the composition does not contain a preservative.

In some embodiments, the antibody comprises a heavy chain constant region, such as for example IgG, IgM, IgD, IgA, and IgE; and any isotypes, such as IgG1, IgG2, IgG3, and IgG4. Preferably, the antibody is an IgG2 or IgG4 antibody. In some embodiments, the antibody comprises a constant region of IgG4 comprising the following mutations (Armour et al., 2003, Molecular Immunology 40 585-593): E233F234L235 to P233V234A235 (IgG4Δc), in which the numbering is with reference to wild type IgG4. In yet another embodiment, the Fc is human IgG4 E233F234L235 to P233V234A235 with deletion G236 (IgG4Δb). In some embodiments the Fc is any human IgG4 Fc (IgG4, IgG4Δb or IgG4Δc) containing hinge stabilizing mutation S228 to P228 (Aalberse et al., 2002, Immunology 105, 9-19).

In some embodiments, the antibody can be selected from the group consisting of monoclonal antibodies, polyclonal antibodies, antibody fragments (e.g., Fab, Fab′, F(ab′)2, Fv, Fc, ScFv etc.), chimeric antibodies, bispecific antibodies, heteroconjugate antibodies, single chain (ScFv), mutants thereof, fusion proteins comprising an antibody portion (e.g., a domain antibody), humanized antibodies, human antibodies, and any other modified configuration of the immunoglobulin molecule that comprises an antigen recognition site of the required specificity, including glycosylation variants of antibodies, amino acid sequence variants of antibodies, and covalently modified antibodies. The antibody may be murine, rat, human, or any other origin (including chimeric or humanized antibodies). In some embodiments, the antibody can be human but is more preferably humanized. Preferably the antibody is isolated, further preferably it is substantially pure. Where the antibody is an antibody fragment this preferably retains the functional characteristics of the original antibody i.e. the ligand binding and/or antagonist or agonist activity.

Illustrative anti-PD-1 antibodies include, but are not limited to, for example: nivolumab (OPDIVO®, ONO-4538, BMS-936558, MDX1106, Bristol-Myers Squibb Company), pembrolizumab (KEYTRUDA®, MK-3475, lambrolizumab, Merck & Co., Inc.), BCD100 (BIOCAD Biopharmaceutical Company), BGB-A317 (BeiGene Ltd./Celgene Corporation), CBT-501 (CBT Pharmaceuticals), GLS-010 (Harbin Gloria Pharmaceuticals Co., Ltd.), 161308 (Innovent Biologics, Inc.), AMP-224 (GlaxoSmithKline plc), BI 754091 (Boehringer Ingelheim GmbH), PDR001 (Novartis AG), MEDI0680 (AstraZeneca PLC), PDR001 (Novartis AG), PF-06801591 (aka RN888) (Pfizer Inc.), described as mAb7 in International Patent Publication No. WO2016/092419, the disclosure of which is hereby incorporated by reference in its entirety, REGN2810 (Regeneron Pharmaceuticals, Inc.), SHR-1210 (Incyte Corporation), TSR-042 (Tesaro, Inc.), AGEN2034 (Agenus Inc.), JNJ-63723283 (Johnson & Johnson), MGD013 (MacroGenics, Inc.), ANA011 (AnaptysBio, Inc.), ANB011 (AnaptysBio, Inc.), AUNP-12 (Pierre Fabre Medicament S.A.), ENUM 244C8 (Enumeral Biomedical Holdings, Inc.), hAb21 (Stainwei Biotech, Inc.), J43 (Transgene S.A.), JTX-4014 (Jounce Therapeutics, Inc.), MCLA-134 (Merus B.V.), PRS-332 (Pieris AG), SHR-1316 (Atridia Pty Ltd.), STI-A1010 (Sorrento Therapeutics, Inc.), STI-A1110 (Les Laboratoires Servier), and XmAb20717 (Xencor, Inc.).

BGB-A317 (tislelizumab), under development by BeiGene Ltd., is a humanized IgG4, monoclonal antibody having an engineered Fc region (i.e., where the ability to bind Fc gamma receptor I has been specifically removed). BGB-A317 binds to PD-1 and inhibits the binding of PD-1 to PD-L1 and PD-L2.

In one or more embodiments, the PD-1 axis binding antagonist is selected from PF-06801591, nivolumab, pembrolizumab, spartalizumab, and BGB-A317.

In a specific aspect, the anti-PD-1 antibody is PF-06801591 (Pfizer Inc., CAS Registry Number 2029210-61-3). In another specific aspect, the anti-PD-1 antibody is nivolumab (OPDIVO®, MDX-1106, CAS Registry Number 946414-94-4). In another specific aspect, the anti-PD-1 antibody is cemiplimab (LIBTAYO®, also known as REGN2810, Regeneron Pharmaceuticals, Inc. and sanofi-aventis U.S. LLC, CAS Registry Number 1801342-60-8). In another specific aspect, the anti-PD-1 antibody is nivolumab (OPDIVO®, MDX-1106, CAS Registry Number 946414-94-4). In another specific aspect, the anti-PD-1 antibody is spartalizumab (PDR001, Novartis, CAS Registry Number 1935694-88-4). In another specific aspect, a PD-1 binding antagonist is the antibody comprising a VH region produced by the expression vector with ATCC Accession No. PTA-121 183 and having the VL region produced by the expression vector with ATCC Accession No. PTA-1 21 182, also known as mAb7 or mAb15 (Rinat Neuroscience, Pfizer Inc.) as described herein.

In a specific aspect, the anti-PD-1 antibody comprises a heavy chain variable region comprising:

(a) a CDR1 comprising the amino acid sequence shown in SEQ ID NO: 4 (GYTFTSYWIN);

(b) a CDR2 comprising the amino acid sequence shown in SEQ ID NO: 5 (NIYPGSSLTNYNEKFKN); and

(c) a CDR3 comprising the amino acid sequence shown in SEQ ID NO: 6 (LSTGTFAY).

In some embodiments, the antibody can be an anti-PD-1 antibody comprising a light chain variable region comprising:

(a) a CDR1 comprising the amino acid sequence shown in SEQ ID NO: 7 (KSSQSLWDSGNQKNFLT);

(b) a CDR2 comprising the amino acid sequence shown in SEQ ID NO: 8 (WTSYRES); and

(c) a CDR3 comprising the amino acid sequence shown in SEQ ID NO: 9 (QNDYFYPHT).

In some embodiments, the antibody can be anti-PD-1 antibody comprising three CDRs from a heavy chain variable region comprising the amino acid sequence shown in SEQ ID NO: 2.

(SEQ ID NO: 2)
QVQLVQSGAEVKKPGASVKVSCKASGYTFTSYWINWVRQAPGQGLEWMGN
IYPGSSLTNYNEKFKNRVTMTRDTSTSTVYMELSSLRSEDTAVYYCARLS
TGTFAYWGQGTLVTVSS

In some embodiments, the antibody can be anti-PD-1 antibody comprising three CDRs from a light chain variable region comprising the amino acid sequence shown in SEQ ID NO: 3.

(SEQ ID NO: 3)
DMMTQSPDSLAVSLGERATINCKSSQSLWDSGNQKNFLTWYQQKPGQPPK
LLIYWTSYRESGVPDRFSGSGSGTDFTLTISSLQAEDVAVYYCQNDYFYP
HTFGGGTKVEIK

In some embodiments, an anti-PD-1 antibody may comprise a heavy chain variable region comprising an amino acid sequence of any of at least about 80%, 85%, 90%, 91%, 92%, 93%, 94%, 95%, 96%, 97%, 98% or 99% A identical to the amino acid sequence comprising the amino acid sequence shown in SEQ ID NO. 2 and/or a light chain variable region comprising an amino acid sequence of any of at least about 80%, 85%, 90%, 91%, 92%, 93%, 94%, 95%, 96%, 97%, 98% or 99% A identical to the amino acid sequence comprising the amino acid sequence shown in SEQ ID NO. 3, wherein the antibody binds specifically to human PD-1.

In some embodiments, an anti-PD-1 antibody may comprise a heavy chain variable region comprising the amino acid sequence comprising the amino acid sequence shown in SEQ ID NO: 2 and/or may comprise a light chain variable region comprising the amino acid sequence comprising the amino acid sequence shown in SEQ ID NO: 3.

In some embodiments, an anti-PD-1 antibody may be an antibody comprising the amino acid sequences shown in SEQ ID NOS: 2 and 3.

In some embodiments, an anti-PD-1 antibody may comprise a heavy chain region comprising an amino acid sequence of any of at least about 80%, 85%, 90%, 91%, 92%, 93%, 94%, 95%, 96%, 97%, 98% or 99% A identical to the amino acid sequence comprising the amino acid sequence shown in SEQ ID NO: 10 and/or a light chain region comprising an amino acid sequence of any of at least about 80%, 85%, 90%, 91%, 92%, 93%, 94%, 95%, 96%, 97%, 98% or 99% A identical to the amino acid sequence comprising the amino acid sequence shown in SEQ ID NO: 11, wherein the antibody binds specifically to human PD-1.

Heavy chain sequence
(SEQ ID NO: 10)
QVQLVQSGAEVKKPGASVKVSCKASGYTFTSYWINWVRQAPGQGLEWMGN
IYPGSSLTNYNEKFKNRVTMTRDTSTSTVYMELSSLRSEDTAVYYCARLS
TGTFAYWGQGTLVTVSSASTKGPSVFPLAPCSRSTSESTAALGCLVKDYF
PEPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLMI
SRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK
Light chain sequence
(SEQ ID NO: 11)
DMMTQSPDSLAVSLGERATINCKSSQSLWDSGNQKNFLTWYQQKPGQPPK
LLIYWTSYRESGVPDRFSGSGSGTDFTLTISSLQAEDVAVYYCQNDYFYP
HTFGGGTKVEIKRGTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC

In some embodiments, an anti-PD-1 antibody may comprise a heavy chain region comprising the amino acid sequence comprising the amino acid sequence shown in SEQ ID NO: 10 and/or may comprise a light chain region comprising the amino acid sequence comprising the amino acid sequence shown in SEQ ID NO: 11. In some embodiments, the C-terminal lysine of the heavy chain sequence shown in SEQ ID NO: 10 can be cleaved; i.e., the heavy chain sequence can lack a C-terminal lysine.

In some embodiments, an anti-PD-1 antibody may be an antibody comprising the amino acid sequences shown in SEQ ID NOS: 10 and 11.

In some embodiments, an anti-PD-1 antibody may compete for PD-1 binding with an anti-PD-1 antibody as defined herein. The anti-PD-1 antibody may compete for PD-1 binding with an antibody comprising a heavy chain variable region comprising the amino acid sequence comprising the amino acid sequence shown in SEQ ID NO: 2 and/or a light chain variable region comprising the amino acid sequence comprising the amino acid sequence shown in SEQ ID NO: 3.

In some embodiments, an anti-PD-1 antibody may be a monoclonal antibody, mAb7, which specifically binds human PD-1. Antibody mAb7 is described in WO2016/092419, the content of which is hereby incorporated by reference in its entirety. The amino acid sequences of the heavy chain and light chain variable regions of mAb7 are shown in SEQ ID NOs: 2 and 3, respectively. The CDR portions of antibody mAb7 (including Chothia and Kabat CDRs) are diagrammatically depicted in Table 1 of WO2016/092419. Antibody mAb7 is highly potent in blocking PD-1 biological activity.

In some embodiments, the anti-PD-1 antibody may also comprise a fragment or a region of the antibody mAb7. In one embodiment, the fragment is a light chain of the antibody mAb7 comprising the amino acid sequence as shown in SEQ ID NO: 11 herein. In another embodiment, the fragment is a heavy chain of the antibody mAb7 comprising the amino acid sequence as shown in SEQ ID NO: 10 herein. In yet another embodiment, the fragment contains one or more variable regions from a light chain and/or a heavy chain of the antibody mAb7. In yet another embodiment, the fragment contains one or more CDRs from a light chain and/or a heavy chain of the antibody mAb7 comprising the amino acid sequences as shown in SEQ ID NOS: 11 and 10, respectively, herein.

In some embodiments, the antibody may comprise one or more (one, two, three, four, five, or six) CDR(s) derived from antibody mAb7. In some embodiments, the CDRs may be Kabat CDRs, Chothia CDRs, or a combination of Kabat and Chothia CDRs (termed “extended” or “combined” CDRs herein). In some embodiments, the polypeptides comprise any of the CDR configurations (including combinations, variants, etc.) described herein.

In some embodiments of the present invention the C-terminal lysine of the heavy chain of any of the anti-PD-1 antibodies described herein is deleted. In various cases the heavy and/or light chain of the anti-PD-1 antibodies described herein may optionally include a signal sequence.

In other embodiments, the antibody may be selected from an anti-PD-1 antibody known in the art, such as antibodies described in, for example without limitation, any of the following: U.S. Pat. Nos. 8,354,509, 9,084,776, 9,492,540, 9,492,539, 9,387,247, 8,779,105, 8,952,136, and 8,709,416. The antibody may bind to the same epitope as an anti-PD-1 antibody known in the art and/or may compete for binding to PD-1 with such an antibody.

According to a further aspect of the present invention there is provided a composition comprising or consisting essentially of;

about 140 mg/ml to about 160 mg/ml of antibody,

about 10.0 mM to about 30.0 mM histidine buffer,

about 40 mg/ml to about 100 mg/ml trehalose,

about 0.01 to about 0.3 mg/ml polysorbate 80 (PS80), and

about 0.01 to about 0.1 mg/ml disodium EDTA,

wherein said composition is of a pH selected from the the range of between about pH 4.5 and any of about pH 5.5, or alternatively from the range of between about pH 4.5 and any of about pH 4.6, 4.7, 4.8, 4.9, 5.0, 5.1, 5.2, 5.3, 5.4, 5.5, 5.6, 5.7, 5.8, 5.9

According to a preferred embodiment the composition comprises or consists essentially of any of about 90 mg/ml, about 100 mg/ml, about 110 mg/ml, about 120 mg/ml, about 130 mg/ml, about 140 mg/ml or about 150 mg/ml of antibody,

about 20 mM histidine buffer,

about 84 mg/ml trehalose,

about 0.2 mg/ml PS80, and

about 0.05 mg/ml disodium EDTA,

wherein said composition is of a pH selected from the the range of between about pH 5.0 and any of about pH 5.0, 5.2, 5.5 or 5.8, or alternatively from the range of between about pH 4.5 and any of about pH 4.5, 4.6, 4.7, 4.8, 4.9, 5.0, 5.1, 5.2, 5.3, 5.4, or 5.5, and wherein said antibody comprises a variable heavy chain sequence comprising the amino acid sequence shown in SEQ ID NO. 2 and a variable light chain sequence comprising the amino acid sequence shown in SEQ ID NO. 3.

According to a preferred embodiment the composition comprises or consists essentially of any of about 120 mg/ml, about 130 mg/ml, about 140 mg/ml, about 150 mg/ml, about 160 mg/ml, about 170 mg/ml or about 180 mg/ml of antibody,

about 20 mM histidine buffer,

about 84 mg/ml trehalose,

about 0.2 mg/ml PS80, and

about 0.05 mg/ml disodium EDTA,

wherein the pH of said composition is about pH 5.0, +/−0.5 and wherein said antibody comprises a variable heavy chain sequence comprising the amino acid sequence shown in SEQ ID NO. 2 and a variable light chain sequence comprising the amino acid sequence shown in SEQ ID NO. 3. In some embodiment the dose volume used is about 0.5 ml, about 1 ml, about 2 ml, about 3 ml, about 4 ml, about 5 ml, about 6 ml, about 7 ml, about 8 ml, about 9 ml, about 10 ml, about 11 ml, about 12 ml, about 13 ml, about 14 ml, about 15 ml, about 16 ml, about 17 ml, about 18 ml, about 19 ml, about 20 ml, about 21 ml, about 22 ml, about 23 ml, about 24 ml, about 25 ml, about 26 ml, about 27 ml, about 28 ml, about 29 ml, about 30 ml, about 31 ml, about 32 ml, about 33 ml, about 34 ml, about 35 ml, about 36 ml, about 37 ml, about 38 ml, about 39 ml, about 40 ml, about 41 ml, about 42 ml, about 43 ml, about 44 ml, about 45 ml, about 46 ml, about 47 ml, about 48 ml, about 49 ml, or about 50 ml.

In some embodiments there is provided a composition which is lyophilized and/or has been subjected to lyophylization. In some embodiments there is provided a composition which is not lyophilized and has not been subjected to lyophylization.

In some embodiments the concentration of antibody is any of about 100 mg/ml, about 105 mg/ml, about 110 mg/ml, about 115 mg/ml, about 120 mg/ml, about 125 mg/ml, about 130 mg/ml, about 135 mg/ml, about 140 mg/ml, about 145 mg/ml, about 150 mg/ml, about 155 mg/ml, or about 160 mg/ml.

According to a further preferred aspect of the present invention there is provided a composition, of any foregoing aspect or embodiment, for the manufacture of a medicament for treatment of a hyperliferative disorder, such as for example cancer, in a subject.

In some embodiments, the cancer is selected from one or more of gastric cancer, sarcoma, lymphoma, Hodgkin's lymphoma, leukemia, head and neck cancer, squamous cell head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma..

According to a yet further embodiment of the invention there is provided a composition, of any foregoing aspect or embodiment, for the manufacture of a medicament for treatment of a hyperliferative disorder, such as for example cancer, in a subject.

According to a preferred embodiment the composition can be administered directly into the blood stream, into muscle, into tissue, into fat, or into an internal organ. Suitable means for parenteral administration include subcutaneous, intravenous, intraarterial, intraperitoneal, intrathecal, intraventricular, intraurethral, intrasternal, intracranial, intramuscular, intra-ossial, and intradermal. Suitable devices for parenteral administration include needle (including microneedle, microprojections, soluble needles and other micropore formation techniques) injectors, needle-free injectors and infusion techniques.

In some embodiments the administration pattern of the medicament comprises administration of a dose of the medicament once every week, once every two weeks, once every three weeks, once every four weeks, once every five weeks, once every six weeks, once every seven weeks, once every eight weeks, once every nine weeks, once every ten weeks, once every fifteen weeks, once every twenty weeks, once every twenty five weeks, or once every twenty six weeks. In some embodiments, the anti-PD-1 antibody is administered once every month, once every two months, once every three months, once every four months, once every five months, or once every six months. In some embodiments the administration pattern of the medicament comprises administration of a dose of the medicament once every four or eight weeks.

In some embodiments the volume of a dose is less than or equal to about 3 ml, about 2.5 ml, about 2 ml, about 1.5 ml, about 1 ml, about 0.75 ml, about 0.5 ml, about 0.25 ml or about 0.1 ml.

In some embodiments the volume of a dose is about 20 ml, about 19 ml, about 18 ml, about 17 ml, about 16 ml, about 15 ml, about 14 ml, about 13 ml, about 12 ml, about 11 ml, about 10 ml, about 9 ml, about 8 ml, about 7 ml, about 6 ml, about 5 ml, about 4 ml, about 3 ml, about 2 ml or about 1 ml. Alternatively, the volume of a dose is about 20.5 ml, about 19.5 ml, about 18.5 ml, about 17.5 ml, about 16.5 ml, about 15.5 ml, about 14.5 ml, about 13.5 ml, about 12.5 ml, about 11.5 ml, about 10.5 ml, about 9.5 ml, about 8.5 ml, about 7.5 ml, about 6.5 ml, about 5.5 ml, about 4.5 ml, about 3.5 ml, about 2.5 ml, about 1.5 ml, or about 0.5 ml. Alternatively, the volume of a dose is about 900 microliters, about 800 microliters, about 700 microliters, about 600 microliters, about 500 microliters, about 400 microliters, about 300 microliters, about 200 microliters, or about 100 microliters, alternatively about 950 microliters, about 850 microliters, about 750 microliters, about 650 microliters, about 550 microliters, about 450 microliters, about 350 microliters, about 250 microliters, about 150 microliters, or about 50 microliters. In some embodiments the volume of the dose is less than or equal to about 2.0 ml.

According to preferred embodiment the concentration of antibody can range from about 0.1 to about 200 mg/ml. Preferably the concentration of antibody is about 100 mg/ml, about 101 mg/ml, about 102 mg/ml, about 103 mg/ml, about 104 mg/ml, about 105 mg/ml, about 106 mg/ml, about 107 mg/ml, about 108 mg/ml, about 109 mg/ml, or about 110 mg/ml, about 111 mg/ml, about 112 mg/ml, about 113 mg/ml, about 114 mg/ml, about 115 mg/ml, about 116 mg/ml, about 117 mg/ml, about 118 mg/ml, about 119 mg/ml, about 120 mg/ml, about 121 mg/ml, about 122 mg/ml, about 123 mg/ml, about 124 mg/ml, about 125 mg/ml, about 126 mg/ml, about 127 mg/ml, about 128 mg/ml, about 129 mg/ml, about 130 mg/ml, about 131 mg/ml, about 132 mg/ml, about 133 mg/ml, about 134 mg/ml, about 135 mg/ml, about 136 mg/ml, about 137 mg/ml, about 138 mg/ml, about 139 mg/ml, about 140 mg/ml, about 141 mg/ml, about 142 mg/ml, about 143 mg/ml, about 144 mg/ml, about 145 mg/ml, about 146 mg/ml, about 147 mg/ml, about 148 mg/ml, about 149 mg/ml, or about 150 mg/ml. Most preferably the concentration of antibody is about 100 mg/ml to about 180 mg/ml and may be selected from the group comprising about 100 mg/ml, about 105 mg/ml, about 110 mg/ml, about 115 mg/ml, about 120 mg/ml, about 125 mg/ml, about 130 mg/ml, about 135 mg/ml, about 140 mg/ml, about 145 mg/ml, about 150 mg/ml, about 155 mg/ml, about 160 mg/ml, about 165 mg/ml, about 170 mg/ml, about 175 mg/ml, or about 180 mg/ml.

According to a preferred embodiment a dose contains less than or equal to about 50 mg, about 51 mg, about 52 mg, about 53 mg, about 54 mg, about 55 mg, about 56 mg, about 57 mg, about 58 mg, about 59 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 850 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, or about 1000 mg of antibody.

According to some embodiments the dose contains an amount of antibody that is about 1 μg/kg, about 10 μg/kg, about 20 μg/kg, about 25 μg/kg, about 50 μg/kg, about 100 μg/kg, about 200 μg/kg, about 250 μg/kg, about 500 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, or about 11 mg/kg (of mass of the subject to which the dose it to be administered). In some embodiments, the dose contains about 20 μg/kg, about 25 μg/kg, about 50 μg/kg, about 100 μg/kg, about 200 μg/kg, about 250 μg/kg, 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg.

Dosage regimens may depend on the pattern of pharmacokinetic decay that the practitioner wishes to achieve. For example, in some embodiments, dosing from one-four times a week is contemplated. Even less frequent dosing may be used. In some embodiments, the dose is administered once every 1 week, every 2 weeks, every 3 weeks, every 4 weeks, every 5 weeks, every 6 weeks, every 7 weeks, every 8 weeks, every 9 weeks, every 10 weeks, every 15 weeks, every 20 weeks, every 25 weeks, or longer. In some embodiments, the dose is administered once every 1 month, every 2 months, every 3 months, every 4 months, every 5 months, every 6 months, or longer. The progress of this therapy is easily monitored by conventional techniques and assays. The dosing regimen can vary over time.

For the purpose of the present invention, the appropriate dosage of the medicament will depend on the antibody employed, the type and severity of the disorder to be treated, whether the agent is administered for preventative or therapeutic purposes, previous therapy, the patient's clinical history and response to the agent, and the discretion of the attending physician. Typically the clinician will administer the medicament, until a dosage is reached that achieves the desired result. Dosages may be determined empirically. For example individuals are given incremental dosages to assess efficacy of the medicament.

Dose and/or frequency can vary over course of treatment. Empirical considerations, such as the antibody half-life, generally will contribute to the determination of the dosage. Frequency of administration may be determined and adjusted over the course of therapy, and is generally, but not necessarily, based on treatment and/or suppression and/or amelioration and/or delay of one or more symptoms of hyperproliferative disease. In some individuals, more than one dose may be required. Frequency of administration may be determined and adjusted over the course of therapy. For example without limitation, for repeated administrations over several days or longer, depending on the disease and its severity, the treatment is sustained until a desired suppression of symptoms occurs or until sufficient therapeutic levels are achieved to treat cancer.

Administration of medicament comprising the composition can be continuous or intermittent, depending, for example, upon the recipient's physiological condition, whether the purpose of the administration is therapeutic or prophylactic, and other factors known to skilled practitioners. The administration of the medicament comprising the composition may be essentially continuous over a preselected period of time or may be in a series of spaced dose.

Preferably the administration of the dose is a parenteral administration preferably selected from intravenous, intraarterial, intraperitoneal, intrathecal, intraventricular, intraurethral, intrasternal, intracranial, intramuscular, intra-ossial, intraderm al and subcutaneous. Preferably the medicament is in a unit dosage sterile form for parenteral administration.

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims. The Examples in WO2016/092419 are referred to illustrate the antibodies for use in the present invention. The entire content of WO2016/092419 is hereby incorporated by reference.

### EXAMPLES

### Methods

This Methods section provides a summary of the methods used in the following Examples 1-6.

The viscosity of antibody formulations was measured on a chip-based m-VROC instrument, in which the pressure difference correlates with solution dynamic viscosity. Measurements were tested at different flow rates and shear rates. Sample size was approximately 70-100 μL. Aliquots were loaded into a 100 μL Hamilton syringe and connected to the m-VROC chip. Triplicate measurements were taken at 20° C.

To determine freeze thaw stability, anti-PD-1 antibody at 100-150 mg/mL was cycled from −20° C. to 2-8° C. for 5 cycles. Antibody stability was evaluated via pH, concentration, and SEC.

To determine agitation stability, anti-PD-1 antibody at high concentration was evaluated after 24 hours of agitation at 300 rpm, and ambient temperature and light in an upright orientation. Stability was evaluated via pH, concentration, and SEC.

The pH stability of high concentration antibody formulations was measured according to USP compendial method <791>and EP compendial method 2.2.3 using a suitable, properly standardized potentiometer instrument (Thermo Scientific Orion Star A111 pH meter) capable of reproducing pH values to 0.02 pH units using an indicator electrode sensitive to hydrogen-ion activity, glass electrode, and suitable reference electrode (Thermo Scientific Orion™ PerpHecT™ ROSS™ Combination pH Micro Electrode). Two- or three-point calibrations at 22.5-25.4° C. were performed on day of use to verify standardization of pH meter, samples equilibrated at ambient temperature for at least 30 minutes, and values to 0.01 pH units recorded after pH reading stabilized for at least 60 seconds.

Total protein concentrations were measuring spectroscopically using SoloVPE (C Technologies Inc.) instrument based on variable path length and Beer-Lambert law (A=clε, where A=absorbance, c=concentration, l=path length, and ε=extinction coefficient/molar absorbance coefficient in mg ml⁻¹ cm⁻¹). For anti-PD-1 antibody mAb7 the ε used is 1.62 mg mg ml⁻¹ cm⁻¹. For nivolumab the ε used is 1.68 mg mg ml⁻¹ cm⁻¹. For pembrolizumab the ε used is 1.42 mg mg ml⁻¹ cm⁻¹. Samples were equilibrated to ambient temperature, added to a sample vessel (C Tech Inc. #OC0009-1-P50), and loaded into the vessel holder in the detection window platform. Per sample, a clean fibrette (#OC0002-P50) was installed into the fibrette coupler, and the slope at 280 nm read (scatter correction at 320 mm).

Formation of higher molecular mass species (HMMS) was analyzed by size exclusion chromatography (SEC) on an Agilent HPLC system. 50 μg of protein was separated based on hydrodynamic volume on an YMC-Pack Diol-200 column (Waters, Cat. No. DL20S053008WT) maintained at 30±2° C. using an isocratic gradient (20 mM sodium phosphate, 400 mM sodium chloride, pH 7.2) at 0.75 mL/min. Molecular weight species were eluted and detected by UV absorption at 280 nm.

The charge heterogeneity and stability of antibodies at high concentration to deamidation and fragmentation was quantified using imaged capillary isoelectric focusing (iCE), which separates protein species based on their charge differences (pl value) in a pH gradient. Samples were diluted in a mixture to final concentration of 0.3 mg/ml protein, 0.01 mg/mL pl marker 6.14 (Protein Simple, part no. 102220), 0.01 mg/mL pl marker 9.50 (Protein Simple, part no. 101996), 4% Pharmalyte pH 3-10 (GE part no. 17-0456-01), 0.25% methyl cellulose (Protein Simple, part no. 101876)), and 2 M urea (Sigma-Aldrich, part no. U4883). Samples were injected into a fluorocarbon coated clEF cartridge column (Protein Simple, part no. 101701) installed in the Protein simple iCE3 instrument, focused in the capillary column under high voltage (≤3000 V), and monitored in a real column imaging detection (WCID) system at a fixed wavelength of 280 nm. The resultant electropherograms are analyzed with appropriate software to determine pl values and peak areas for each species.

Subvisible particles were analyzed using micro-flow imaging (MFI). Before any sample analysis, a 15 μm Duke latex count standard was run as a system suitability check. Water flushes were used between each analysis. In addition, water blank was analyzed before to ensure the background counts were appropriate for testing. The average cumulative counts per ml were reported. In addition, multiple size channels were monitored to provide information on particle counts per size range.

### Example 1. Impact of Buffer and pH on Viscosity

This example illustrates the impact of pH on viscosity in a high concentration anti-PD-1 antibody formulation.

To prepare the antibody formulation, anti-PD-1 antibody mAb7 was buffer exchanged into 20 mM Histidine pH 5.5, 20 mM Histidine pH 6.0, 20 mM Histidine pH 6.5, 20 mM Histidine pH 7.0, 20 mM Acetate pH 5.0, and 20 mM Acetate pH 6.0 by repeatedly diluting with buffer followed by concentrating to approximately 160 mg/mL until buffer exchange was complete. The final concentration of all samples was targeted to be 160 mg/mL.

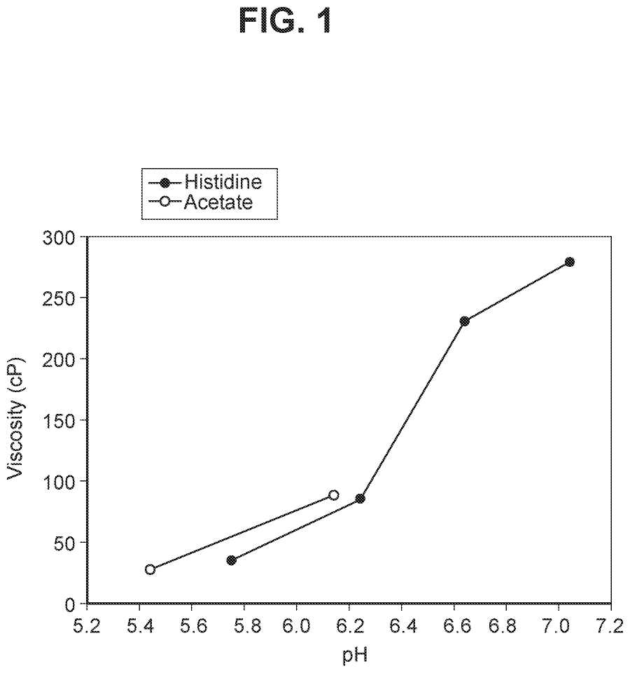

Protein concentration, viscosity, and pH were measured for all samples. Results are summarized in Table 1 and FIG. 1. The results demonstrate that formulations with a lower pH have a significantly lower viscosity. Additionally, at the same pH, histidine and acetate based formulations both have similar viscosities.

TABLE 1

| | PD-1 antibody | Viscosity | |
|---|---|---|---|
| Formulation | Concentration | at 20° C. | Measured |
| (Target pH) | (mg/mL) | (Cp) | pH |
| 20 mM Histidine pH 5.5 | 159.7 | 35.2 | 5.75 |
| 20 mM Histidine pH 6.0 | 161.2 | 85.2 | 6.24 |
| 20 mM Histidine pH 6.5 | 160.9 | 230.9 | 6.64 |
| 20 mM Histidine pH 7.0 | 152.6 | 279.1 | 7.04 |
| 20 mM Acetate pH 5.0 | 156.4 | 27.9 | 5.44 |
| 20 mM Acetate pH 6.0 | 162.5 | 88.6 | 6.14 |

### Example 2. Evaluation of Arginine

This example illustrates the impact of varying concentrations of arginine on the viscosity of anti-PD-1 antibody.

To evaluate the impact of arginine on viscosity, anti-PD-1 antibody mAb7 was formulated into 20 mM Acetate pH 5.0, 50 g/L sucrose, 0.05 g/L Na₂EDTA dihydrate, and 0.2 g/L PS80 by dialyzing (without PS80) with 20 kDa MWCO dialysis cassettes, concentrating to approximately 200 mg/mL anti-PD-1 antibody with 50 kDa Am icon centrifugal filters, and spiking in high concentration PS80. Formulations with varying concentrations of arginine were made by additions of a high concentration arginine hydrochloride (HCl) solution.

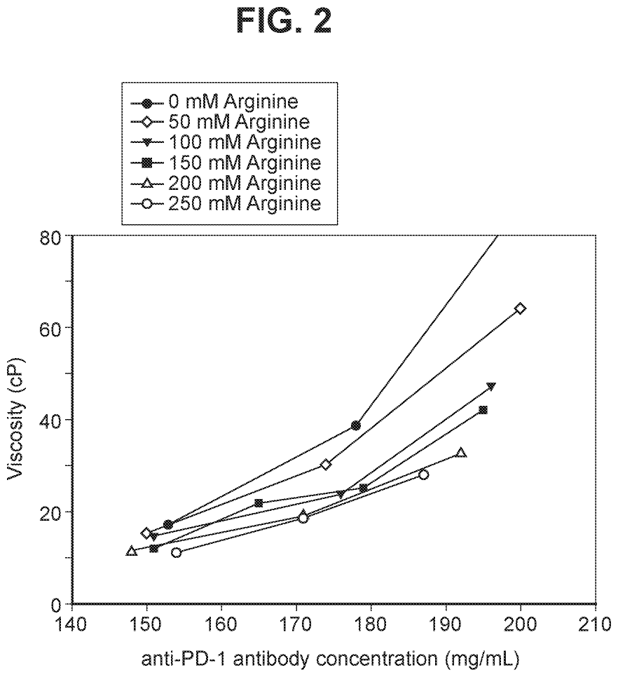

Protein concentration and pH were measured for all samples. Results are summarized in Table 2 and FIG. 2. Viscosities of anti-PD-1 antibody formulations with 0, 50, 100, 150, 200, and 250 mM arginine at a range of anti-PD-1 antibody concentrations are shown (Table 2, FIG. 2). The results demonstrate that the addition of 50 mM arginine decreases the viscosity of high concentration anti-PD-1 antibody formulations. Additionally, incremental increasing the concentration of arginine up to 250 mM further lowers the viscosity.

TABLE 2

| | Anti-PD-1 Antibody | Viscosity at |
|---|---|---|
| Arginine (mM) | Concentration (mg/mL) | 20° C. (cP) |
| 0 | 153 | 17.0 |
| | 178 | 38.6 |
| | 205 | 97.0 |
| 50 | 150 | 15.0 |
| | 174 | 30.1 |
| | 200 | 64.1 |
| 100 | 151 | 14.5 |
| | 176 | 23.7 |
| | 196 | 47.3 |
| 150 | 151 | 11.7 |
| | 179 | 25.0 |
| | 195 | 42.0 |
| | 165 | 21.7 |
| 200 | 148 | 11.2 |
| | 171 | 18.9 |
| | 192 | 32.5 |
| 250 | 154 | 11.0 |
| | 171 | 18.5 |
| | 187 | 27.8 |

### Example 3. Evaluation of Arginine Containing Formulations

This example illustrates the impact of additional excipients on the viscosity and stability of anti-PD-1 antibody.

To prepare the arginine-containing antibody formulations, anti-PD-1 antibody mAb7 was formulated into each of the formulations 1-6 (Table 3) by dialyzing mAb7 into each formulation (without PS80) with 20 kDa MWCO dialysis cassettes, concentrating to 150-200 mg/mL anti-PD-1 antibody with 50 kDa Amicon centrifugal filters, and spiking in high concentration PS80.

TABLE 3

| Formulation | | | Na₂EDTA | | Arginine | |
|---|---|---|---|---|---|---|
| Number | Buffer/pH | Sucrose | dihydrate | PS80 | HCl | Proline |
| 1 | 20 mM | 50 g/L | 0.05 g/L | 0.2 g/L | 150 mM | None |
| | Acetate pH 4.5 | | | | | |
| 2 | 20 mM | 50 g/L | 0.05 g/L | 0.2 g/L | 150 mM | None |
| | Acetate pH 5.0 | | | | | |
| 3 | 20 mM | 50 g/L | 0.05 g/L | 0.2 g/L | 150 mM | None |
| | Acetate pH 5.5 | | | | | |
| 4 | None pH 5.0 | None | None | 0.2 g/L | 100 mM | 200 mM |
| 5 | 20 mM | 50 g/L | 0.05 g/L | 0.2 g/L | 100 mM | 100 mM |
| | Acetate pH 5.0 | | | | | |
| 6 | 20 mM | 50 g/L | 0.05 g/L | 0.2 g/L | 100 mM | None |
| | Acetate pH 5.0 | | | | | |

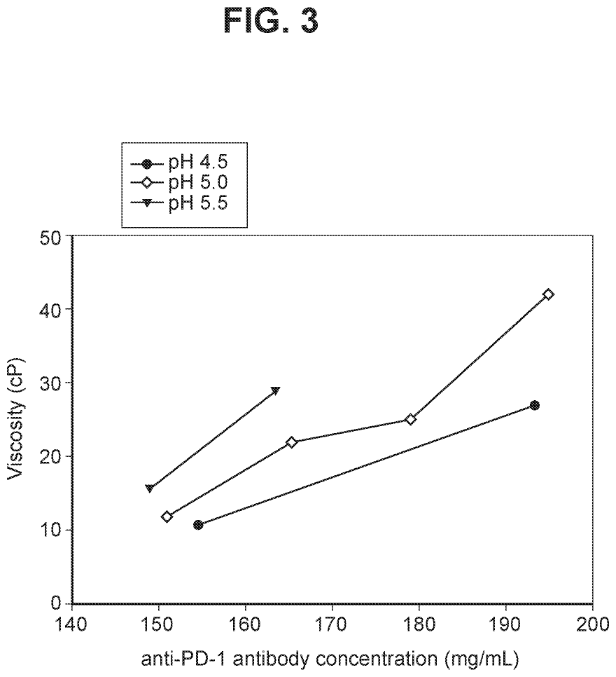

Formulations 1, 2, and 3 evaluate the impact of pH in arginine-containing formulations. Table 4 and FIG. 3 summarize the viscosity of formulations 1, 2, and 3 at a range of anti-PD-1 concentrations. The results demonstrate that in formulations containing 150 mM arginine, lowering the pH from 5.5 to 4.5 significantly lowers the viscosity.

TABLE 4

| | Antibody | Viscosity at |
|---|---|---|
| Formulation | Concentration (mg/ml) | 20° C. (cP) |
| 1 | 154.6 | 10.5 |
| | 193.3 | 26.8 |
| 2 | 151 | 11.7 |
| | 179 | 25 |
| | 195 | 42 |
| | 165.4 | 21.7 |
| 3 | 149 | 15.6 |
| | 163.5 | 28.9 |

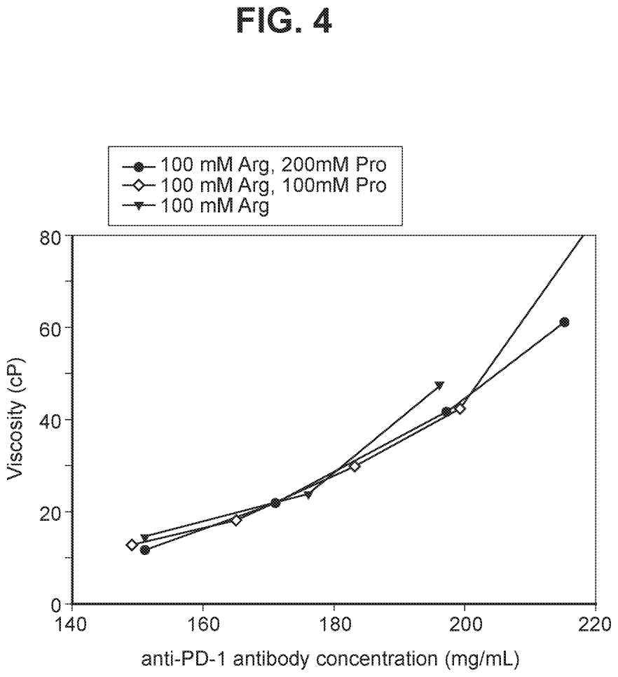

Formulations 4, 5, and 6 evaluated the impact of the addition of proline to formulations containing 100 mM Arginine at pH 5.0. Table 5 and FIG. 4 summarize the viscosity of formulations 4, 5, and 6 at a range of anti-PD-1 antibody concentrations. The results demonstrate that there is no significant difference to the viscosity of high concentration anti-PD-1 antibody formulations containing 0, 100, or 200 mM proline.

TABLE 5

| | Antibody | Viscosity at |
|---|---|---|
| Formulation | Concentration (mg/mL) | 20° C. (cP) |
| 4 | 151 | 11.6 |
| | 171 | 21.7 |
| | 197 | 41.4 |
| | 215 | 60.9 |
| 5 | 149 | 12.6 |
| | 165 | 18.0 |
| | 183 | 29.8 |
| | 199 | 42.0 |
| | 230 | 104.0 |
| 6 | 151 | 14.5 |
| | 176 | 23.7 |
| | 196 | 47.3 |

The stability of formulations 1, 2, 3, 4, and 5 at 150 mg/mL anti-PD-1 antibody were determined by placing samples on stability at 5° C., 25° C., and 40° C. Protein stability was assessed with regard to aggregation (SEC), charge isoforms (iCE), concentration, and pH. Tables 6, 7, 8, 9, and 10 summarize the thermal stability of formulations 1, 2, 3, 4, and 5, respectively.

TABLE 6
Formulation 1 Temperature Stability

| | | 5° C. | | 25° C. | | | 40° C. | | |
|---|---|---|---|---|---|---|---|---|---|
| Test | T = 0 | 4 W | 10 W | 2 W | 4 W | 10 W | 2 W | 4 W | 10 W |
| Anti-PD-1 Antibody | 154.6 | 156.0 | 154.1 | NS | 155.0 | 156.7 | NS | 153.6 | 162.9 |
| Concentration (mg/mL) | | | | | | | | | |
| pH | 4.5 | 4.5 | 4.5 | 4.5 | 4.5 | 4.5 | 4.6 | 4.6 | 4.7 |
| SEC: Total HMMS (%) | 0.6 | 0.6 | 0.6 | NS | 0.8 | 1.0 | NS | 2.6 | 1.9 |
| ICE: Acidic (%) | 25.5 | 23.4 | 23.8 | 26.0 | 24.5 | 28.5 | 20.8 | 19.3 | 11.6 |
| ICE: Basic (%) | 12.9 | 13.4 | 16.6 | 14.4 | 14.5 | 17.3 | 40.3 | 49.4 | 68.3 |
| ICE: Main (%) | 61.6 | 62.4 | 59.6 | 59.6 | 61.0 | 54.2 | 39.0 | 31.2 | 16.2 |

TABLE 7
Formulation 2 Temperature Stability

| | | 5° C. | | 25° C. | | | 40° C. | | |
|---|---|---|---|---|---|---|---|---|---|
| Test | T = 0 | 4 W | 10 W | 2 W | 4 W | 10 W | 2 W | 4 W | 10 W |
| Anti-PD-1 Antibody | 151.0 | 149.7 | 148.6 | NS | 150.3 | 151.7 | NS | 151.0 | 152.6 |
| Concentration (mg/mL) | | | | | | | | | |
| pH | 5.0 | 5.0 | 5.0 | 5.0 | 5.0 | 5.0 | 5.0 | 5.0 | 5.0 |
| SEC: Total HMMS (%) | 0.6 | 0.6 | 0.6 | NS | 0.7 | 0.8 | NS | 2.3 | 2.4 |
| ICE: Acidic (%) | 24.4 | 22.9 | 24.3 | 26.7 | 23.6 | 27.9 | 29.1 | 31.3 | 40.6 |
| ICE: Basic (%) | 13.1 | 13.8 | 14.5 | 13.0 | 14.3 | 17.0 | 17.6 | 16.3 | 22.2 |
| ICE: Main (%) | 62.5 | 63.4 | 61.2 | 60.3 | 62.0 | 55.1 | 53.3 | 52.4 | 37.2 |

TABLE 8
Formulation 3 Temperature Stability

| | | 5° C. | | 25° C. | | | 40° C. | | |
|---|---|---|---|---|---|---|---|---|---|
| Test | T = 0 | 4 W | 10 W | 2 W | 4 W | 10 W | 2 W | 4 W | 10 W |
| Anti-PD-1 Antibody | 149.0 | 153.4 | 149.9 | NS | 148.7 | 147.6 | NS | 148.4 | 156.0 |
| Concentration (mg/mL) | | | | | | | | | |
| pH | 5.5 | 5.5 | 5.5 | 5.5 | 5.5 | 5.5 | 5.5 | 5.5 | 5.5 |
| SEC: Total HMMS (%) | 0.6 | 0.6 | 0.6 | NS | 0.7 | 0.7 | NS | 1.3 | 2.2 |
| ICE: Acidic (%) | 27.1 | 23.9 | 24.5 | 25.8 | 23.2 | 24.9 | 30.3 | 33.7 | 46.0 |
| ICE: Basic (%) | 13.9 | 13.5 | 14.6 | 14.3 | 14.2 | 16.4 | 15.8 | 14.0 | 13.1 |
| ICE: Main (%) | 59.0 | 62.6 | 60.9 | 59.9 | 62.6 | 58.7 | 54.0 | 52.3 | 41.0 |

TABLE 9
Formulation 4 Temperature Stability

| | | 5° C. | 25° C. | | 40° C. | |
|---|---|---|---|---|---|---|
| Test | T = 0 | 4 W | 2 W | 4 W | 2W | 4 W |
| Anti-PD-1 Antibody | 151.5 | 154.2 | NS | 150.6 | NS | 154.0 |
| Concentration (mg/mL) | | | | | | |
| pH | 5.1 | 5.0 | 5.1 | 5.0 | 5.1 | 5.1 |
| SEC: Total HMMS (%) | 1.0 | 0.9 | NS | 1.0 | NS | 2.5 |
| ICE: Acidic (%) | 26.5 | 23.1 | 25.8 | 22.4 | 29.1 | 31.2 |
| ICE: Basic (%) | 12.9 | 14.0 | 14.1 | 14.5 | 17.5 | 16.9 |
| ICE: Main (%) | 60.6 | 62.9 | 60.1 | 63.1 | 53.4 | 51.8 |

TABLE 10
Formulation 5 Temperature Stability

| | | 5° C. | 25° C. | | 40° C. | |
|---|---|---|---|---|---|---|
| Test | T = 0 | 4 W | 2 W | 4 W | 2 W | 4 W |
| Anti-PD-1 Antibody | 148.8 | 148.1 | NS | 145.9 | NS | 148.6 |
| Concentration (mg/mL) | | | | | | |
| pH | 5.0 | 5.0 | 5.1 | 5.0 | 5.0 | 5.0 |
| SEC: Total HMMS (%) | 0.6 | 0.6 | NS | 0.7 | NS | 2.0 |
| ICE: Acidic (%) | 21.9 | 23.1 | 25.4 | 23.7 | 29.6 | 32.5 |
| ICE: Basic (%) | 12.3 | 13.8 | 15.2 | 14.4 | 16.7 | 17.5 |
| ICE: Main (%) | 65.8 | 63.0 | 59.4 | 61.8 | 53.7 | 50.0 |

For all formulations 1-5, there is no significant change after 10 weeks (4 weeks for formulations 4 and 5) to the charge species and aggregates (HMMS) at 5° C. and 25° C. At 40° C., formulation 1 (pH 4.5) had a significant increase in basic species whereas formulations 2, 3, 4, and 5 (pH>4.5) had an increase in acidic species. Aggregates (HMMS) increased a similar amount for all formulations after storage at 40° C., however the increase is considered acceptable for liquid formulations with intended storage conditions of 5° C.

### Example 4. Evaluation of Trehalose-Containing Formulations

This example illustrates the effect trehalose on stability and viscosity of anti-PD-1 antibody formulations.

Trehalose dihydrate was evaluated as a stabilizer in arginine-free and arginine-containing formulations (Table 11).

TABLE 11

| Formulation | | Trehalose | Na₂EDTA | | Arginine |
|---|---|---|---|---|---|
| Number | Buffer/pH | Dihydrate | dihydrate | PS80 | HCl |
| 7 | 20 mM | 84 g/L | 0.05 g/L | 0.2 g/L | None |
| | Histidine pH | | | | |
| | 5.0 | | | | |
| 8 | 20 mM | 50 g/L | 0.05 g/L | 0.2 g/L | 100 mM |
| | Histidine pH | | | | |
| | 5.0 | | | | |

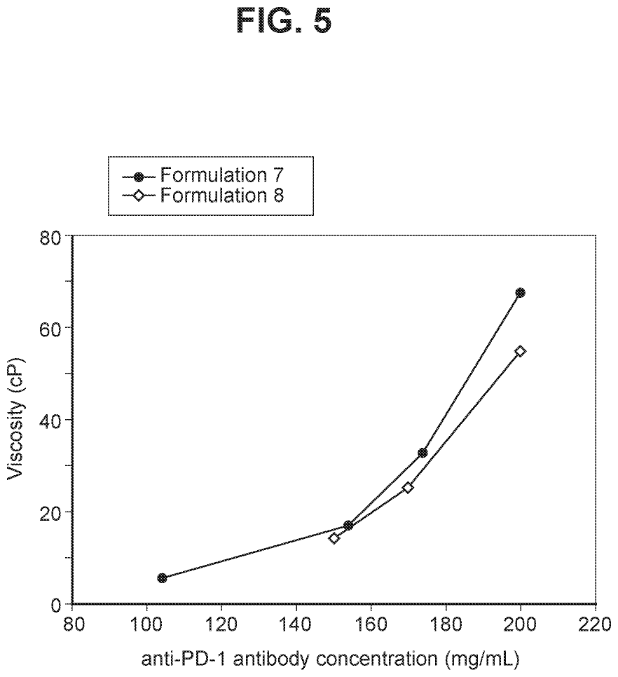

To prepare the trehalose-containing antibody formulations, anti-PD-1 antibody mAb7 was buffer exchanged into 20 mM histidine and 50 g/L trehalose dihydrate using tangentrial flow filtration with 50 kDa ultrafiltration cartridges. The anti-PD-1 antibody mAb7 concentration was increased using ultrafiltration, followed by addition of excipients using high concentration solutions, and dilution as necessary. Anti-PD-1 antibody mAb7 was formulated in formulation 7 at the following concentrations: 104 mg/ml, 154 mg/ml, 174 mg/ml, and 200 mg/ml. Anti-PD-1 antibody mAb7 was formulated in formulation 8 at the following concentrations: 150 mg/ml, 170 mg/ml, and 200 mg/ml. Viscosity was measured as described above. Results are summarized in Table 12 and FIG. 5.

TABLE 12
Viscosities of Trehalose Containing Formulations

| | Antibody | Viscosity at |
|---|---|---|
| Formulation | concentration (mg/mL) | 20° C. (cP) |
| 7 | 104 | 5.3 |
| | 154 | 16.9 |
| | 174 | 32.6 |
| | 200 | 67.6 |
| 8 | 150 | 14.1 |
| | 170 | 25.0 |
| | 200 | 54.4 |

Viscosity of formulation 7 containing 104 mg/ml, 154 mg/ml, 174 mg/ml, and 200 mg/ml mAb7 is 5.3, 16.9, 32.5, and 67.6 cP, respectively at 20° C. Viscosity of formulation 8 containing 150 mg/ml, 170 mg/ml, and 200 mg/ml mAb7 is 14.1, 25.0, and 54.4 cP, respectively at 20° C.

The results demonstrate that formulation 8 (containing arginine) has a lower viscosity than formulation 7 at anti-PD-1 antibody concentrations ≥175 g/L. Viscosity of formulation 7 and 8 at about 150 mg/ml was similar (FIG. 5).

The stabilities of the anti-PD-1 antibody formulations 7 and 8 were assessed at 5° C., 25° C., and 40° C. Formulation 7 was tested using 100 mg/mL and 150 mg/mL anti-PD-1 antibody, and formulation 8 using 150 mg/mL anti-PD-1 antibody. Samples were place at 5° C., 25° C., and 40° C., and stability was assessed by measuring aggregation (SEC), charge isoforms (iCE), concentration, pH, and subvisible particulates (MFI) as described above. Results are summarized in Tables 13-21 (NT=not tested).

Tables 13, 14, and 15 summarize thermal stability for formulation 7 at 150 mg/mL anti-PD-1 at 5° C., 25° C., and 40° C., respectively.

TABLE 13
Formulation 7 (150 mg/mL) Temperature Stability 5° C.

| Test | T0 | T2 W | T4 W | T6 W | T10 W | T14 W |
|---|---|---|---|---|---|---|
| Anti-PD-1 Antibody | 154.2 | NT | 153.5 | 156.5 | 153.8 | 154.1 |
| Concentration | | | | | | |
| (mg/mL) | | | | | | |
| pH | 5.1 | NT | 5.0 | 5.0 | 5.0 | 5.0 |
| SEC: Total HMMS | 0.6 | NT | 0.6 | 0.6 | 0.6 | 0.6 |
| (%) | | | | | | |
| ICE: Acidic (%) | 23.0 | NT | 24.2 | 23.8 | 26.4 | 26.2 |
| ICE: Basic (%) | 13.1 | NT | 12.5 | 13.9 | 12.2 | 13.3 |
| ICE: Main (%) | 63.9 | NT | 63.3 | 62.3 | 61.3 | 60.5 |
| MFI: Cumulative ≥ | 330 | NT | 38 | 8 | 57 | 11 |
| 10 μm (particle/mL) | | | | | | |
| MFI: Cumulative ≥ | 83 | NT | 0 | 4 | 11 | 8 |
| 25 μm (particle/mL) | | | | | | |

TABLE 14
Formulation 7 (150 mg/mL) Temperature Stability 25° C.

| Test | T0 | T2 W | T4 W | T6 W | T10 W | T14 W |
|---|---|---|---|---|---|---|
| Anti-PD-1 Antibody | 154.2 | 150.4 | 154.1 | 156.0 | 155.1 | 153.9 |
| Concentration | | | | | | |
| (mg/mL) | | | | | | |
| pH | 5.1 | 5.1 | 5.0 | 5.0 | 5.1 | 5.0 |
| SEC: Total HMMS | 0.6 | 0.6 | 0.7 | 0.7 | 0.8 | 0.9 |
| (%) | | | | | | |
| ICE: Acidic (%) | 23.0 | 22.7 | 24.5 | 24.9 | 29.9 | 29.6 |
| ICE: Basic (%) | 13.1 | 15.5 | 12.9 | 13.8 | 13.5 | 13.9 |
| ICE: Main (%) | 63.9 | 61.8 | 62.5 | 61.3 | 56.6 | 56.5 |
| MFI: Cumulative ≥ | 330 | 23 | 11 | 76 | 92 | 31 |
| 10 μm (particle/mL) | | | | | | |
| MFI: Cumulative ≥ | 83 | 4 | 4 | 0 | 23 | 8 |
| 25 μm (particle/mL) | | | | | | |

TABLE 15
Formulation 7 (150 mg/mL) Temperature Stability 40° C.

| Test | T0 | T2 W | T4 W | T6 W | T10 W | T14 W |
|---|---|---|---|---|---|---|
| Anti-PD-1 Antibody | 154.2 | 152.6 | 156.7 | 154.2 | 154.6 | NT |
| Concentration | | | | | | |
| (mg/mL) | | | | | | |
| pH | 5.1 | 5.1 | 5.1 | 5.1 | 5.1 | NT |
| SEC: Total HMMS | 0.6 | 1.1 | 1.8 | 2.2 | 3.4 | NT |
| (%) | | | | | | |
| ICE: Acidic (%) | 23.0 | 28.5 | 35.0 | 39.0 | 49.5 | NT |
| ICE: Basic (%) | 13.1 | 15.9 | 15.8 | 15.6 | 13.4 | NT |
| ICE: Main (%) | 63.9 | 55.7 | 49.2 | 45.3 | 37.1 | NT |
| MFI: Cumulative ≥ | 330 | 23 | 157 | 23 | 88 | NT |
| 10 μm (particle/mL) | | | | | | |
| MFI: Cumulative ≥ | 83 | 0 | 4 | 0 | 11 | NT |
| 25 μm (particle/mL) | | | | | | |

Tables 16, 17, and 18 summarize thermal stability for formulation 7 at 10 mg/mL anti-PD-1 at 5° C., 25° C., and 40° C., respectively.

TABLE 16
Formulation 7 (100 mg/mL) Temperature Stability 5° C.

| Test | T0 | T2 W | T4 W | T6 W | T10 W | T14 W |
|---|---|---|---|---|---|---|
| Anti-PD-1 Antibody | 104.4 | NT | 104.7 | 104.2 | 107.0 | 104.0 |
| Concentration | | | | | | |
| (mg/mL) | | | | | | |
| pH | 5.0 | NT | 5.0 | 5.1 | 5.0 | 5.0 |
| SEC: Total HMMS | 0.6 | NT | 0.6 | 0.6 | 0.6 | 0.6 |
| (%) | | | | | | |
| ICE: Acidic (%) | 23.2 | NT | 28.2 | 25.0 | 27.7 | 26.4 |
| ICE: Basic (%) | 13.0 | NT | 12.3 | 13.5 | 13.1 | 13.6 |
| ICE: Main (%) | 63.8 | NT | 59.5 | 61.5 | 59.1 | 60.0 |
| MFI: Cumulative ≥ | 84 | NT | 126 | 19 | 19 | 15 |
| 10 μm (particle/mL) | | | | | | |
| MFI: Cumulative ≥ | 4 | NT | 11 | 0 | 8 | 8 |
| 25 μm (particle/mL) | | | | | | |

TABLE 17
Formulation 7 (100 mg/mL) Temperature Stability 25° C.

| Test | T0 | T2 W | T4 W | T6 W | T10 W | T14 W |
|---|---|---|---|---|---|---|
| Anti-PD-1 Antibody | 104.4 | 103.4 | 104.0 | 104.5 | 104.0 | 104.6 |
| Concentration | | | | | | |
| (mg/mL) | | | | | | |
| pH | 5.0 | 5.1 | 5.0 | 5.0 | 5.0 | 5.0 |
| SEC: Total HMMS | 0.6 | 0.6 | 0.6 | 0.7 | 0.7 | 0.7 |
| (%) | | | | | | |
| ICE: Acidic (%) | 23.2 | 23.1 | 27.2 | 25.6 | 30.7 | 29.7 |
| ICE: Basic (%) | 13.0 | 14.8 | 12.6 | 13.7 | 13.3 | 13.6 |
| ICE: Main (%) | 63.8 | 62.2 | 60.2 | 60.7 | 56.0 | 56.7 |
| MFI: Cumulative ≥ | 84 | 11 | 0 | 23 | 183 | 23 |
| 10 μm (particle/mL) | | | | | | |
| MFI: Cumulative ≥ | 4 | 4 | 0 | 8 | 11 | 4 |
| 25 μm (particle/mL) | | | | | | |

TABLE 18
Formulation 7 (100 mg/mL) Temperature Stability 40° C.

| Test | T0 | T2 W | T4 W | T6 W | T10 W | T14 W |
|---|---|---|---|---|---|---|
| Anti-PD-1 Antibody | 104.4 | 104.3 | 105.7 | NT | NT | NT |
| Concentration | | | | | | |
| (mg/mL) | | | | | | |
| pH | 5.0 | 5.0 | 5.0 | 5.0 | 5.0 | NT |
| SEC: Total HMMS | 0.6 | 1.0 | 1.5 | 1.9 | 3.2 | NT |
| (%) | | | | | | |
| ICE: Acidic (%) | 23.2 | 27.6 | 36.3 | 39.5 | 49.1 | NT |
| ICE: Basic (%) | 13.0 | 18.5 | 14.7 | 15.8 | 13.5 | NT |
| ICE: Main (%) | 63.8 | 53.9 | 49.0 | 44.6 | 37.5 | NT |
| MFI: Cumulative ≥ | 84 | 31 | 643 | 27 | 50 | NT |
| 10 μm (particle/mL) | | | | | | |
| MFI: Cumulative ≥ | 4 | 4 | 46 | 0 | 8 | NT |
| 25 μm (particle/mL) | | | | | | |

Tables 19, 20, and 21 summarize thermal stability for formulation 8 at 150 mg/mL anti-PD-1 at 5° C., 25° C., and 40° C., respectively.

TABLE 19
Formulation 8 (150 mg/mL) Temperature Stability 5° C.

| Test | T0 | T2 W | T4 W | T6 W | T10 W | T14 W |
|---|---|---|---|---|---|---|
| Anti-PD-1 Antibody | 148.8 | NT | 151.0 | 150.4 | NT | NT |
| Concentration | | | | | | |
| (mg/mL) | | | | | | |
| pH | 5.1 | NT | 5.1 | 5.1 | NT | NT |
| SEC: Total HMMS | 0.6 | NT | 0.6 | 0.6 | NT | NT |
| (%) | | | | | | |
| ICE: Acidic (%) | 22.8 | NT | 27.3 | 24.8 | NT | NT |
| ICE: Basic (%) | 12.8 | NT | 12.6 | 13.2 | NT | NT |
| ICE: Main (%) | 64.4 | NT | 60.1 | 62.0 | NT | NT |
| MFI: Cumulative ≥ | 508 | NT | 61 | 23 | NT | NT |
| 10 μm (particle/mL) | | | | | | |
| MFI: Cumulative ≥ | 37 | NT | 8 | 8 | NT | NT |
| 25 μm (particle/mL) | | | | | | |

TABLE 20
Formulation 8 (150 mg/mL) Temperature Stability 25° C.

| Test | T0 | T2 W | T4 W | T6 W | T10 W | T14 W |
|---|---|---|---|---|---|---|
| Anti-PD-1 Antibody | 148.8 | 152.0 | 153.8 | 152.0 | NT | NT |
| Concentration | | | | | | |
| (mg/mL) | | | | | | |
| pH | 5.1 | 5.1 | 5.1 | 5.1 | NT | NT |
| SEC: Total HMMS | 0.6 | 0.6 | 0.7 | 0.7 | NT | NT |
| (%) | | | | | | |
| ICE: Acidic (%) | 22.8 | 24.1 | 25.1 | 24.6 | NT | NT |
| ICE: Basic (%) | 12.8 | 14.9 | 13.7 | 13.7 | NT | NT |
| ICE: Main (%) | 64.4 | 61.1 | 60.1 | 61.7 | NT | NT |
| MFI: Cumulative ≥ | 508 | 332 | 2637 | 134 | NT | NT |
| 10 μm (particle/mL) | | | | | | |
| MFI: Cumulative ≥ | 37 | 4 | 573 | 11 | NT | NT |
| 25 μm (particle/mL) | | | | | | |

TABLE 21
Formulation 8 (150 mg/mL) Temperature Stability 40° C.

| Test | T0 | T2 W | T4 W | T6 W | T10 W | T14 W |
|---|---|---|---|---|---|---|
| Anti-PD-1 Antibody | 148.8 | 149.0 | 152.8 | 153.0 | NT | NT |
| Concentration | | | | | | |
| (mg/mL) | | | | | | |
| pH | 5.1 | 5.1 | 5.0 | 5.1 | NT | NT |
| SEC: Total HMMS | 0.6 | 1.1 | 2.0 | 1.9 | NT | NT |
| (%) | | | | | | |
| ICE: Acidic (%) | 22.8 | 27.9 | NT | 37.8 | NT | NT |
| ICE: Basic (%) | 12.8 | 18.0 | NT | 16.1 | NT | NT |
| ICE: Main (%) | 64.4 | 54.1 | NT | 46.2 | NT | NT |
| MFI: Cumulative ≥ | 508 | 4 | 27 | 65 | NT | NT |
| 10 μm (particle/mL) | | | | | | |
| MFI: Cumulative ≥ | 37 | 0 | 8 | 8 | NT | NT |
| 25 μm (particle/mL) | | | | | | |

No significant change in protein concentration, pH, or subvisible particulates was observed for all formulations at all studied conditions (Tables 13-21). Additionally, no significant change in aggregation or charge species at 5° C. or 25° C. was observed. Aggregates (HMMS) and acidic species increases for all formulations after storage at 40° C., however the increase is the same in all formulations and the relative increase is considered acceptable for liquid formulations with an intended storage condition of 5° C.

These results demonstrate that formulation 7 containing 150 mg/ml anti-PD-1 antibody mAb7, 20 mM histidine, 84 mg/ml trehalose dihydrate, 0.05 mg/ml disodium EDTA dihydrate, and 0.2 mg/ml PS80, at pH 5.0-5.1, is stable after 14 weeks of storage at 5° C. or 25° C. (Tables 13 and 14). These results also demonstrate that formulation 7 containing 100 mg/ml anti-PD-1 antibody mAb7, 20 mM histidine, 84 mg/ml trehalose dihydrate, 0.05 mg/ml disodium EDTA dihydrate, and 0.2 mg/ml PS80, at pH 5.0-5.1, is stable after 14 weeks of storage at 5° C. or 25° C. (Tables 16 and 17). These results also demonstrate that formulation 8 containing 150 mg/ml anti-PD-1 antibody mAb7, 20 mM histidine, 100 mM arginine, 50 mg/ml trehalose dihydrate, 0.05 mg/ml disodium EDTA dihydrate, and 0.2 mg/ml PS80 at pH 5.0, is stable after 6 weeks of storage at 5° C. or 25° C. (Tables 19 and 20).

The freeze-thaw and agitation stability of formulation 7 at 100 and 150 mg/mL anti-PD-1 and of formulation 8 at 150 mg/mL anti-PD-1 were determined by stressing the formulations to either 5 freeze/thaw (FT) cycles or 24 hours of agitation (AG). Results are summarized in Table 22.

TABLE 22
Formulation 7 (100 mg/mL and 150 mg/mL) and Formulation
8 Freeze/Thaw and Agitation Stability

| | Formulation 7 (150 mg/mL) | | | Formulation 7 (100 mg/mL) | | | Formulation 8 (150 mg/mL) | | |
|---|---|---|---|---|---|---|---|---|---|
| Test | Initial | AG | FT | Initial | AG | FT | Initial | AG | FT |
| Anti-PD-1 | 154.2 | 152.9 | 152.1 | 104.4 | 104.4 | 102.8 | 148.8 | 148.7 | 151.7 |
| Antibody | | | | | | | | | |
| Concentration | | | | | | | | | |
| (mg/mL) | | | | | | | | | |
| pH | 5.1 | 5.0 | 5.1 | 5.0 | 5.0 | 5.0 | 5.1 | 5.1 | 5.1 |
| SEC: Total | 0.6 | 0.6 | 0.6 | 0.6 | 0.6 | 0.6 | 0.6 | 0.6 | 0.6 |
| HMMS (%) | | | | | | | | | |

Similarly low levels of aggregate formation after stressing by either freeze/thaw or agitation were observed for formulation 7 and formulation 8, i.e. 0.6%, (Table 22).

These results demonstrate that forumulation 7 containing 100 or 150 mg/ml anti-PD-1 antibody mAb7, 20 mM histidine, 84 mg/ml trehalose dihydrate, 0.05 mg/ml disodium EDTA dihydrate, and 0.2 mg/ml PS80, at pH 5.0-5.1, is stable after 5 freeze/thaw cycles or 24 hours of agitation. Forumulation 8 containing 150 mg/ml anti-PD-1 antibody mAb7, 20 mM histidine, 100 mM arginine HCl, 50 mg/ml trehalose dihydrate, 0.05 mg/ml disodium EDTA dihydrate, 0.2 mg/ml PS80, pH 5.0) is also stable after 5 freeze/thaw cycles or 24 hours of agitation.

### Example 5. Glycosylation pattern of anti-PD-1 antibody mAb7

This example illustrates the glycosylation pattern of anti-PD-1 antibody mAb7.

Peptide mapping by LC/MS confirmed one site of N-glycosylation located on the heavy chain peptide containing the N²⁹⁴ST consensus sequence. The N²⁹⁴ST consensus sequence is essentially fully occupied. The N-linked oligosaccharide profile observed for mAb7 displays two major N-glycans, G0F and G1F, both of which are core-fucosylated, complex-type biantennary structures. In addition, less abundant N-glycans, corresponding to truncated and/or afucosylated complex-type biantennary structures, high mannose-type ManS structure and sialylated, core-fucosylated complex-type biantennary oligosaccharides, also are detected and identified.

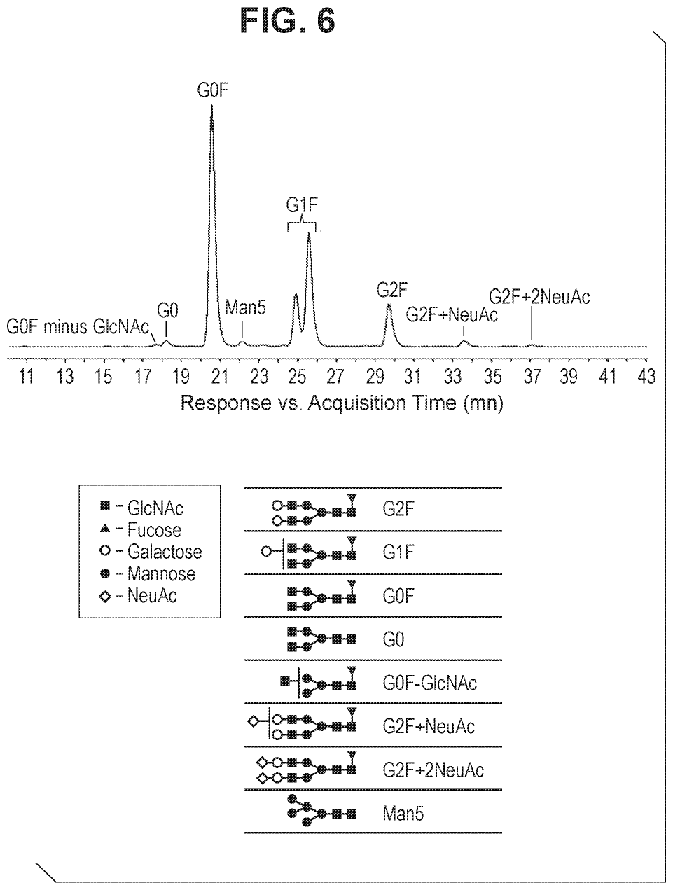

N-linked oligosaccharide profiling of mAb7 involved 2-aminobenzamide (2-AB) labeling of N-linked oligosaccharides released by peptide-N-glycosidase F (PNGaseF). The 2-AB labeled N-linked oligosaccharides were separated by hydrophilic interaction liquid chromatography (HILIC) with fluorescence detection and structural elucidation by mass spectrometry. The glycan heterogeneity in mAb7 is shown in the graph in FIG. 6.

### Example 6. Evaluation of Anti-PD-1 antibodies in Formulation 7

This example evaluates the feasibility of the use of formulation 7 with KEYTRUDA® (pembrolizumab) and OPDIVO® (nivolumab).

Pembrolizumab and nivolumab were each formulated in 20 mM histidine buffer, 84 mg/ml trehalose dihydrate, pH 5.0. High concentration polysborate 80 and disodium EDTA dihydrate were spiked into the samples for a final formulation of 20 mM histidine, 84 mg/ml trehalose dihydrate, 0.05 mg/ml disodium EDTA dihydrate, and 0.2 mg/ml PS80, at pH 5.0 (formulation 7). All formulations were then filtered using 0.22 um PES filters and diluted to required concentrations using protein free formulation 7 solution.

Pembrolizumab was formulated in formulation 7 at 129, 150, 175, and 193. mg/mL. Nivolumab was formulated in formulation 7 at 125, 148, and 179 mg/mL Viscosity at 20° C. was measured as described above. Results are summarized in Table 23.

TABLE 23
Viscosities of Anti-PD-1 Antibodies in Formulation 7

| | Antibody | Viscosity at |
|---|---|---|
| Antibody | concentration (mg/mL) | 20° C. (cP) |
| Pembrolizumab | 129 | 7.1 |
| | 150 | 11.7 |
| | 175 | 24.2 |
| | 193 | 41.4 |
| Nivolumab | 125 | 7.1 |
| | 148 | 13.9 |
| | 179 | 35.1 |

The results demonstrate that the viscosities as a function of protein concentration of pembrolizumb and nivolumab in formulation 7 are comparable the viscosity of anti-PD-1 antibody mAb7 in formulation 7 (Table 12).

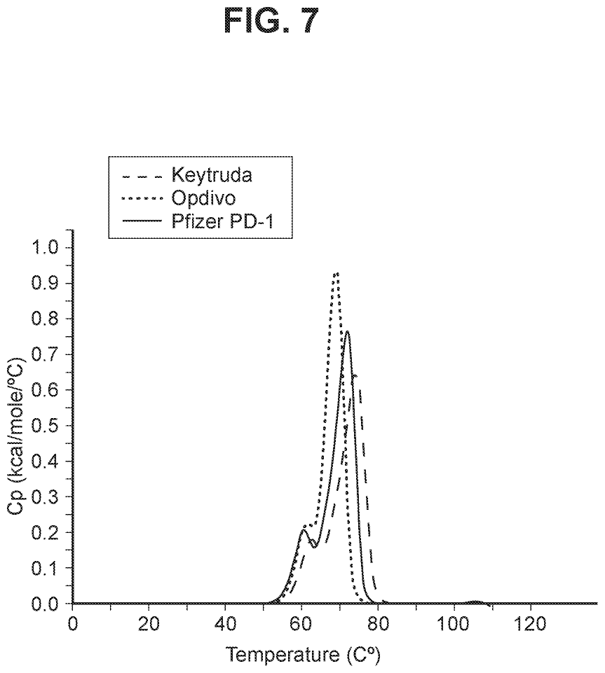

The thermal stability of pembrolizumb, nivolumab, and anti-PD-1 antibody mAb7 in formulation 7 was evaluated by measuring the melting temperature by differential scanning calorimetry. All three antibodies were diluted to 1 mg/mL using protein free formulation 7 solution prior to analysis. A thermal scan was performed from 10° C. to 110° C. at a 100° C./hour ramp rate. The thermograms are shown in FIG. 7 and the onset of thermal unfolding (Tₒₙₛₑₜ) and melting temperatures (T_{m1,m,2}) are shown in Table 24.

TABLE 24
Thermal Properties of Anti-PD-1 Antibodies in Formulation 7

| Antibody | Tₒₙₛₑₜ (° C.) | Tₘ₁ (° C.) | Tₘ₂ (° C.) |
|---|---|---|---|
| Pembrolizumab | 52.2 | 62.5 | 74.1 |
| Nivolumab | 52.7 | 61.4 | 69.0 |
| anti-PD-1 | 51.8 | 60.5 | 71.9 |
| antibody mAb7 | | | |

The thermal stability results demonstrate that pembrolizumb, nivolumab, and anti-PD-1 antibody mAb7 in formulation 7 have comparable thermal profiles, melting onset tempeatures, and melting tempeatures.

The stabilities of pembrolizumb and nivolumab at 150 mg/mL in formulation 7 were assessed at 40° C. for 2 weeks by measuring aggregation (SEC), charge isoforms (iCE), concentration, purity by reduced capillary gel electrophoresis (rCGE), and pH. Results are summarized in Tables 25 and 26.

TABLE 25
Formulation 7 (150 mg/mL pembrolizumab)
Temperature Stability 40° C.

| Test | T0 | T2W |
|---|---|---|
| Pembrolizumb Antibody | 154.0 | 150.7 |
| Concentration (mg/mL) | | |
| pH | 5.0 | 5.1 |
| SEC: Total HMMS (%) | 0.3 | 1.5 |
| ICE: Acidic (%) | 28.3 | 26.9 |
| ICE: Basic (%) | 13.4 | 16.4 |
| ICE: Main (%) | 53.6 | 54.6 |
| rCGE: Purity (%) | 99.6 | 99.2 |

TABLE 26
Formulation 7 (150 mg/mL nivolumab)
Temperature Stability 40° C.

| Test | T0 | T2W |
|---|---|---|
| Nivolumab Antibody | 150.2 | 153.3 |
| Concentration (mg/mL) | | |
| pH | 5.1 | 5.2 |
| SEC: Total HMMS (%) | 0.7 | 1.9 |
| ICE: Acidic (%) | 36.9 | 38.3 |
| ICE: Basic (%) | 5.1 | 12.7 |
| ICE: Main (%) | 54.7 | 47.9 |
| rCGE: Purity (%) | 99.0 | 99.1 |

These results demonstrate that formulation 7 containing 150 mg/ml pembrolizumb, 20 mM histidine, 84 mg/m 1 trehalose dihydrate, 0.05 mg/m 1 disodium EDTA dihydrate, and 0.2 mg/m 1 PS80, at pH 5.0-5.1, is stable after 2 weeks of storage at 40° C. These results also demonstrate that formulation 7 containing 150 mg/ml nivolumab, 20 mM histidine, 84 mg/ml trehalose dihydrate, 0.05 mg/ml disodium EDTA dihydrate, and 0.2 mg/m 1 PS80, at pH 5.1-5.2, is stable after 2 weeks of storage at 40° C.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. A pharmaceutical composition comprising;
an anti-PD-1 antibody, wherein the antibody concentration is between about 100 mg/ml to about 300 mg/ml;
a disaccharide;
a buffer;
a chelating agent; and
a polysorbate,
wherein the pH of said pharmaceutical composition is from about 4.5 to about 5.5, and wherein said pharmaceutical composition has a viscosity of between about 1 centiPoise (cP) and about 20 cP.

2. The pharmaceutical composition of claim 1, wherein the polysorbate is polysorbate 80 (PS80).

3. The pharmaceutical composition of claim 1, wherein the concentration of polysorbate is from about 0.01 to about 0.3 mg/ml.

4. The pharmaceutical composition of claim 3, wherein the pharmaceutical composition comprises 0.2 mg/ml PS80.

5. The pharmaceutical composition of claim 1, wherein the buffer is a histidine buffer.

6. The pharmaceutical composition of claim 5, wherein the concentration of histidine is about 20 mM.

7. The pharmaceutical composition of claim 1, wherein the buffer is an acetate buffer.

8. The pharmaceutical composition of claim 1, wherein the chelating agent is EDTA, and/or wherein the concentration of chelating agent ranges from about 0.01 to about 0.3 mg/mL.

9. The pharmaceutical composition of claim 8, wherein the EDTA comprises disodium EDTA, disodium EDTA dihydrate, or a combination of disodium EDTA and disodium EDTA dihydrate.

10. The pharmaceutical composition of claim 9, wherein the concentration of EDTA is about 0.04 mg/mL, about 0.045 mg/mL, or about 0.05 mg/mL.

11. The pharmaceutical composition of claim 1, wherein the disaccharide is sucrose.

12. The pharmaceutical composition of claim 1, wherein the disaccharide is trehalose.

13. The pharmaceutical composition of claim 12, wherein the trehalose is trehalose dihydrate.

14. The pharmaceutical composition of claim 1, wherein the concentration of disaccharide is between about 25 mg/mL to about 100 mg/mL, about 50 mg/mL or about 84 mg/mL.

15. The pharmaceutical composition of any claim 1, further comprising arginine at a concentration of between about 25 mM to about 300 mM, about 50 mM, about 100 mM, about 150 mM, about 200 mM, or about 250 mM.

16. The pharmaceutical composition of claim 1, further comprising proline.

17. The pharmaceutical composition of claim 16, wherein the concentration of proline is between about 25 mM to about 300 mM, or about 100 mM or about 200 mM.

18. The pharmaceutical composition of claim 1, wherein the antibody concentration is selected from the group consisting of about 140 mg/ml, about 145 mg/ml, about 150 mg/ml, about 155 mg/ml, about 160 mg/ml, about 165 mg/ml, about 170 mg/ml, about 175 mg/ml, about 180 mg/ml, about 185 mg/ml, about 190 mg/ml, about 195 mg/ml, and about 200 mg/ml.

19. The pharmaceutical composition of claim 1, wherein the antibody concentration is about 140 mg/ml to about 200 mg/ml, 145 mg/ml to about 160 mg/ml, or about 148 mg/ml to about 152 mg/ml.

20. The pharmaceutical composition of claim 1, comprising or consisting of:
about 150 mg/ml anti-PD-1 antibody;
about 20 mM histidine buffer;
about 84 mg/ml trehalose;
about 0.2 mg/ml PS80; and
about 0.45 or about 0.5 mg/ml EDTA,
wherein said pharmaceutical composition is pH 5.0+/−0.5.

21. The pharmaceutical composition of claim 20, wherein the viscosity of the composition is between about 10 cP and about 18 cP at at 20° C.

22. The pharmaceutical composition of claim 21, wherein the viscosity of the composition is about 15 cP at 20° C.

23. The pharmaceutical composition of claim 1, comprising or consisting essentially of:
about 150 mg/ml anti-PD-1 antibody;
about 20 mM histidine buffer;
about 100 mM arginine HCl;
about 50 mg/ml trehalose;
about 0.2 mg/ml PS80; and
about 0.45 or about 0.5 mg/ml EDTA,
wherein said pharmaceutical composition is pH 5.0+/−0.5.

24. The pharmaceutical composition of claim 23, wherein the viscosity of the composition is between about 10 cP and about 18 cP at at 20° C.

25. The pharmaceutical composition of claim 24, wherein the viscosity of the composition is about 15 cP at 20° C.

26. The pharmaceutical composition of claim 20, wherein the EDTA comprises disodium EDTA, disodium EDTA dihydrate, or a combination of disodium EDTA and disodium EDTA dihydrate.

27. The pharmaceutical composition of claim 1, wherein the antibody is a human or humanized monoclonal antibody.

28. The pharmaceutical composition of claim 1, wherein the antibody is an IgG4 antibody.

29. The pharmaceutical composition of claim 28, wherein the antibody is an IgG4 S228P antibody.

30. The pharmaceutical composition of claim 1, wherein the antibody comprises a heavy chain variable region (VH) comprising a VH complementarity determining region one (CDR1), a VH CDR2, and a VH CDR3 of the VH sequence shown in SEQ ID NO: 2; and/or a light chain variable region (VL) comprising a VL CDR1, a VL CDR2, and a VL CDR3 of the VL sequence shown in SEQ ID NO: 3.

31. The pharmaceutical composition of claim 30, wherein the VH CDR1 comprises the amino acid sequence shown in SEQ ID NO: 4, the VH CDR2 comprises the amino acid sequence shown in SEQ ID NO: 5, and the VH CDR3 comprises the amino acid sequence shown in SEQ ID NO: 6, the VL CDR1 comprises the amino acid sequence shown in SEQ ID NO: 7, the VL CDR2 comprises the amino acid sequence shown in SEQ ID NO: 8, and the VL CDR3 comprises the amino acid sequence shown in SEQ ID NO: 9.

32. The pharmaceutical composition of claim 1, wherein the antibody comprises an amino acid sequence that is at least 90% identical to a heavy chain variable region amino acid sequence shown in SEQ ID NO: 2, and an amino acid sequence that is at least 90% identical to a light chain variable region amino acid sequence shown in SEQ ID NO: 3.

33. The pharmaceutical composition of claim 1, wherein the antibody comprises a heavy chain variable region (VH) comprising the amino acid sequence shown in SEQ ID NO: 2, or a variant with one or several conservative amino acid substitutions in residues that are not within a CDR and/or a light chain variable region (VL) comprising the amino acid sequence shown in SEQ ID NO: 3, or a variant thereof with one or several amino acid substitutions in amino acids that are not within a CDR.

34. The pharmaceutical composition of claim 1, wherein the antibody comprises a heavy chain comprising the amino acid sequence shown in SEQ ID NO: 10, with or without the C-terminal lysine of SEQ ID NO: 10; and a light chain comprising the amino acid sequence shown in SEQ ID NO: 11.

35. The pharmaceutical composition of claim 34, wherein the antibody displays glycosylation at Asn²⁹⁴ comprising G1F and G1F as the main glycan species.

36. The pharmaceutical composition of claim 35, wherein the glycosylation further comprises as minor glycan species truncated and/or afucosylated complex-type biantennary structures, a high mannose-type Man5 structure, and sialylated, core-fucosylated complex-type biantennary oligosaccharides.

37. The pharmaceutical composition of claim 1, wherein the pharmaceutical composition is lyophilized or is not lyophilized.

38. The pharmaceutical composition of claim 1, wherein the pharmaceutical composition has a viscosity of about 10 to about 18 cP at 20° C.

39. The pharmaceutical composition of claim 1, wherein the antibody is PF-06801591, nivolumab, pembrolizumab, cemiplimab, or spartalizumab, or an antigen binding portion of any of the forgoing.

40. A pharmaceutical composition comprising or consisting essentially of:
about 150 mg/ml anti-PD-1 antibody, wherein the antibody comprises a heavy chain comprising the amino acid sequence shown in SEQ ID NO: 10, with or without the C-terminal lysine of SEQ ID NO: 10; and a light chain comprising the amino acid sequence shown in SEQ ID NO: 11;
about 20 mM histidine buffer;
about 84 mg/ml trehalose;
about 0.2 mg/ml PS80; and
about 0.45 or about 0.5 mg/ml EDTA,
wherein said pharmaceutical composition is pH 5.0+/−0.5 and has a viscosity of of about 10 to about 18 cP at 20° C.

41. A pharmaceutical composition comprising or consisting essentially of:
about 150 mg/ml anti-PD-1 antibody, wherein the antibody comprises a heavy chain comprising the amino acid sequence shown in SEQ ID NO: 10, with or
without the C-terminal lysine of SEQ ID NO: 10; and a light chain comprising the amino acid sequence shown in SEQ ID NO: 11;
about 20 mM histidine buffer;
about 84 mg/ml trehalose;
about 0.2 mg/ml PS80; and
about 0.45 or about 0.5 mg/ml EDTA,
wherein said pharmaceutical composition is pH 5.0+/−0.5 and has a viscosity of of about 10 to about 18 cP at 20° C.

42. The pharmaceutical composition of claim 40, wherein the EDTA comprises disodium EDTA, disodium EDTA dihydrate, or a combination of disodium EDTA and disodium EDTA dihydrate.

43. The pharmaceutical composition of claim 40, wherein the trehalose is trehalose dihydrate.

44. The pharmaceutical composition of claim any one of claim 1, wherein the pharmaceutical composition does not comprise an anti-oxidant.

45. The pharmaceutical composition of claim 44, wherein the anti-oxidant is L-methionine, or a pharmaceutically acceptable salt thereof.

46. The pharmaceutical composition of claim 1, wherein the pharmaceutical composition does not comprise methionine.

47. The pharmaceutical composition of claim 1, wherein the pharmaceutical composition does not comprise arginine.

48. A method of treating a disease, comprising administering an effective amount of the pharmaceutical composition of claim 1 to a subject having such disease.

49. The method of claim 48, wherein the pharmaceutical composition comprises 150 mg/mL anti-PD-1 antibody.

50. A method for treating cancer in a subject in need thereof, the method comprising administering to the subject (1) an effective amount of the pharmaceutical composition of claim 1, and (2) an effective amount of a vaccine capable of eliciting an immune response against cells of the cancer.

51. A method for enhancing the immunogenicity or therapeutic effect of a vaccine administered to a subject for the treatment of cancer, the method comprising administering to the subject receiving the vaccine an effective amount of the pharmaceutical composition of claim 1.

52. The method of claim 48, wherein the pharmaceutical composition is administered as a single 2 mL subcutaneous injection.

53. The method of claim 48, wherein the pharmaceutical composition is administered once every three weeks.

54. The method of claim 48, wherein the pharmaceutical composition is administered once every four weeks.

55. The method of claim 48, wherein the pharmaceutical composition is administered at a dose of 300 mg subcutaneously.

56. The method of claim 48, wherein the subject is administered at least one other therapeutic agent selected from the group consisting of: crizotinib, palbociclib, talazoparib, an anti-CTLA4 antibody, an anti-4-1BB antibody, an anti-OX40 antibody, a second PD-1 antibody, a CD40 agonist, a TLR agonist, a CAR-T cell, and a chemotherapeutic agent.

57. The method of claim 48, wherein the disease is cancer.

58. The method of claim 57, wherein the cancer is selected from the group consisting of gastric cancer, sarcoma, lymphoma, Hodgkin's lymphoma, leukemia, head and neck cancer, squamous cell head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma.

59-63. (canceled)
